(19) United States
(12) Patent Application Publication
(10) Pub. No.: US 2008/0189048 A1
(43) Pub. Date: Aug. 7, 2008

(54) SEQUENCING OF OLIGONUCLEOTIDES BY MASS SPECTROMETRY

(75) Inventors: Heather Desaire, Lawrence, KS (US); Mary Bandu, Eudora, KS (US)

Correspondence Address: WORKMAN NYDEGGER, 60 EAST SOUTH TEMPLE, 1000 EAGLE GATE TOWER, SALT LAKE CITY, UT, 84111, US

(73) Assignee: UNIVERSITY OF KANSAS, Lawrence, KS (US)

(21) Appl. No.: 11/945,114

(22) Filed: Nov. 26, 2007

Related U.S. Application Data
(60) Provisional application No. 60/861,598, filed on Nov. 29, 2006.

(51) Int. Cl. G01N 33/48; B01D 59/44

(52) U.S. Cl. 702/20; 250/282

(57) ABSTRACT

A method of sequencing nucleic acids is provided which uses ion ratios derived by measuring ion abundance at both a high and low collision energy. These ion rations are useful for assigning charge states to product ions, as well as for sequencing the terminal sequences. In addition, methods for using internal ions to sequence the nucleic acids are described.

### CROSS-REFERENCE TO RELATED APPLICATIONS

This U.S. patent application claims the benefit of U.S. provisional patent application having Ser. No. 60/861,598, entitled “SEQUENCING OF OLIGONUCLEOTIDES BY MASS SPECTROMETRY” filed on Nov. 29, 2006, with Mary L. Bandu and Heather Desaire as inventors, which U.S. provisional patent application is incorporated herein in its entirety by specific reference.

### BACKGROUND OF THE INVENTION

Since the recognition of nucleic acids such as DNA as the carrier of the genetic code, a great deal of interest has centered around determining the sequence of that code in the many forms in which it is found. In particular, circulating nucleic acids in plasma and serum have gained considerable attention since their discovery in 1948. Several techniques have been developed for the characterizing circulating DNA. While the Sanger method of DNA analysis is the current “industry standard,” other more high-throughput approaches are in high demand. One very attractive platform for rapid DNA analysis, therefore, is implementing mass spectrometry (“MS”) to sequence the DNA. See Nordhoff et al., Mass spectrometry of nucleic acids, Mass Spectrom. Rev. 15 (1996) 67-138; Murray, DNA sequencing by mass spectrometry, J. Mass Spectrom. 31 (1996) 1203-1215; Banoub et al., Recent Developments in mass spectrometry for the characterization of nucleosides, nucleotides, oligonucleotides, and nucleic acids, Chem. Rev. 105 (2005) 1869-1915; Huber, Analysis of nucleic acids by on-line liquid chromatography-mass spectrometry, Mass Spectrom. Rev. 20 (2001).

In most examples that implement MS to determine information about DNA, small regions of DNA have been amplified by PCR and analyzed using Electrospray Ionization Mass Spectrometry (“ESI-MS”) to differentiate between the known, normal sequences and sequences containing single nucleotide polymorphisms (“SNPs”). See Muhammad et al., Electrospray ionization quadrupole time-of-flight mass spectrometry and quadrupole mass spectrometry for genotyping single nucleotide substitutions in intact polymerase chain reaction products in K-ras and p53, Rapid Commun. Mass Spectrom. 16 (2002) 2278-2285; Walters et al., Genotyping single nucleotide polymorphisms using intact polymerase chain reaction products by Electrospray quadruple mass spectrometry, Rapid Commun. Mass Spectrom. 15 (2001) 1752-1759; Krahmer et al., MS for identification of single nucleotide polymorphisms and MS/MS for Discrimination of isomeric PCR products, Anal. Chem. 72 (2000) 4033-4040. Differences are determined by mass measurements in the full MS spectrum. In addition, PCR amplification of relatively small regions of bacterial DNA have been used in the same manner. Regions of bacterial DNA (80 to 120 base pairs) have been amplified to identify specific bacterial species, and the mass measurement of the PCR product is compared to known species specific sequences. See Muddiman et al., Characterization of PCR products from Bacilli using electrospray ionization FTICR mass spectrometry, Anal. Chem. 68 (1996) 3705-3712; Krahmer et al., Electrospray quadrupole mass spectrometry analysis of model oligonucleotides and polymerase chain reaction products: determination of base substitutions, nucleotide additions/deletions, and chemical modifications, Anal. Chem. 71 (1999) 2893-2900; Wunschel et al., Heterogeneity in Bacillus cereus PCR products detected by ESI-FTICR mass spectrometry, Anal. Chem. 70 (1998) 1203-1207; Mayr et al., Identification of bacteria by polymerase chain reaction followed by liquid chromatography-mass spectrometry, Anal. Chem. 77 (2005) 4563-4570. Recent research has also demonstrated that tandem mass spectrometry (“MS/MS”) can be used for sequencing PCR products. See Little et al., Sequencing 50-mer DNAs using Electrospray tandem mass spectrometry and complementary fragmentation methods, J. Am. Chem. Soc. 117 (1995) 6783-6784; Little et al., Sequence information from 42-108-mer DNAs (complete for a 50-mer) by tandem mass spectrometry, J. Am. Chem. Soc. 118 (1996) 9352-9359.

Two groups have reported on developing methods to sequence DNA. Both use computer algorithms to interpret the MS/MS data. COMPAS, developed by Huber and associates, compares experimental MS/MS data to predicted product ions generated by the computer for known sequences. See Oberacher et al., Re-sequencing of multiple single nucleotide polymorphisms by liquid chromatography-electrospray ionization mass spectrometry, Nucleic Acids Res. 30 (2002) e67; Oberacher et al., Comparative Sequencing of nucleic acids by liquid chromatography-tandem mass spectrometry, Anal. Chem. 74 (2002) 211-218. The second method, developed by McCloskey, generates possible [w] and [a-Base] ions and compares these possible sequences to experimental MS/MS data. Several possible sequences can be generated and scored for the most accurate fit with the experimental data. Both research groups cite the presence of overlapping signal, from multiple ions with the same m/z value, and secondary backbone cleavages as potential problems in determining sequence from experimental data. See also Ni et al., Interpretation of oligonucleotide mass spectra for determination of sequence using electrospray ionization and tandem mass spectrometry, Anal. Chem. 68 (1996) 1989-1999.

To compete with the “industry standard” Sanger method in sequencing DNA, a method that is low in cost would be ideal. The quadrupole ion trap (“QIT”) mass spectrometer, therefore, provides a very attractive alternative platform for sequencing oligonucleotides because of its relatively low cost. Thus, although a potentially valuable alternative to gel based separations as used in the Sanger method, the QIT has a key drawback as well. It is a low resolution instrument which can be detrimental in MS/MS data analysis of oligonucleotides. See Schwartz et al., Quadrupole ion trap mass spectrometry, Methods Enzymol. 270 (1996) 552-586; Premstaller et al., Factors determining the performance of triple quadrupole, quadrupole ion trap and sector field mass spectrometers in Electrospray ionization tandem mass spectrometry of oligonucleotides. 1. Comparison of performance characteristics, Rapid Commun. Mass Spectrom. 15 (2001) 1045-1052; McLuckey et al., Tandem Mass Spectrometry of Small, Multiply Charged Oligonucleotides, J. Am. Soc. Mass Spectrom. 3 (1992) 60-70. Poor resolution inhibits charge state assignments for DNA product ions in MS/MS data. Without charge state information, the mass of product ions also cannot be determined, resulting in the inability to sequence unknowns. Poor resolution also has a significant effect on mass accuracy, which can make it difficult to assign base compositions to MS peaks. This critical issue of resolution must be addressed, to significantly advance the state of the art in DNA sequencing via affordable mass spectrometric methods.

Therefore, it would be advantageous to have a method of using tandem mass spectrometry experiments in order to overcome any poor resolution. Additionally, it would be advantageous to use the Statistical Test of Equivalent Pathways (“STEP”) analysis with ion abundances in tandem mass spectrometry experiments in order to obtain genealogy information about product ions present in mass spectra. Also, it would be advantageous to obtain genealogy information by calculating STEP ratios by comparing the relative abundances of product ions in tandem mass spectrometry experiments. Furthermore, it would be advantageous to identify the type (e.g., primary or secondary) of all the product ions in an MS/MS experiment.

### BRIEF SUMMARY OF THE INVENTION

In one embodiment, the present invention is a method of determining a 5′ terminal base sequence of a parent nucleic acid having a known mass. Such a method includes: obtaining a first tandem mass spectrum of said parent nucleic acid using a first collision energy; determining an ion abundance of each product ion of interest from said first mass spectrum above a predetermined ion abundance; obtaining a second tandem mass spectrum of said parent nucleic acid using a second collision energy that is different from said first collision energy; determining an ion abundance of each product ion of interest from said second mass spectrum above said predetermined ion abundance; determining an ion ratio defined by the formula: ion ratio=(ion abundance of said product ion of interest in a mass spectrum having a higher collision energy)/(ion abundance of said product ion of interest in a mass spectrum having a lower collision energy); determining a mass difference between the known mass of the parent nucleic acid and a mass of a product ion of interest having a low ion ratio; and comparing the obtained mass difference to a predetermined mass value associated with a known 5′ terminal base sequence, wherein the mass difference provides an indication of the 5′ terminal base sequence.

In one embodiment, the 5′ terminal base sequence comprises a single nucleotide selected from C, T, A, or G, and said predetermined mass value is about 220, 225, 234, or 250 Da, respectively, and wherein a mass difference substantially equal to the predetermined mass value of one of the known 5′ terminal base sequence indicates the 5′ terminal base sequence is the known 5′ base sequence.

In one embodiment, the method further includes: ranking said ion ratios for each product ion of interest from a lowest to a highest ion ratio; determining the mass difference between the parent nucleic acid and at least the product ion of interest having the lowest ion ratio in said ranking; and comparing the mass difference to said predetermined mass value associated with the known 5′ terminal base sequence, wherein the mass difference being substantially the same as the predetermined mass value associated with the known 5′ terminal base sequence identifies the 5′ terminal base sequence of the parent nucleic acid. Alternatively, the method further includes: identifying said ion ratios for each product ion of interest; and determining the mass differences between the parent nucleic acid and selected product ions of interest from a lowest ion ratio to a highest ion ratio until obtaining a mass difference that is substantially the same as the predetermined mass value of one of the known 5′ terminal base sequence.

In one embodiment, the method further includes: determining a total product ion area from said first mass spectrum; determining a product ion area for each product ion of interest from said first mass spectrum; determining the ion abundance of said product ion of interest in said first mass spectrum according to the formula: ion abundance=(product ion area/total product ion area of first mass spectrum); determining a total product ion area from said second mass spectrum; determining a product ion area for each product ion of interest from said second mass spectrum; determining an ion abundance of said product ion of interest in said second mass spectrum according to the formula: ion abundance=(product ion area/total product ion area of second mass spectrum); and wherein said ion ratio is determined according to the formula: ion ratio=((Product ion area)/(Total product ion area of the mass spectrum having the higher collision energy))/((Product ion area)/(Total product ion area of the mass spectrum having the lower collision energy)).

In one embodiment, the total product ion area and product ion area are calculated to:

$$\begin{matrix}Total Product Ion Area = {\sum }_{Lowest m / z}^{2000} m / z relative abundance & ( 1 ) \\ Product Ion Area = {\sum }_{- 1.0}^{+ 1.0} m / z relative abundance ( ion of interest ) & ( 2 )\end{matrix}$$

In one embodiment, the present invention provides a method of determining a 3′ terminal base sequence of a parent nucleic acid. Such a method includes: obtaining a first tandem mass spectrum of said parent nucleic acid using a first collision energy; determining an ion abundance of each product ion of interest from said first mass spectrum above a predetermined ion abundance; obtaining a second tandem mass spectrum using a second collision energy that is different from said first collision energy; determining an ion abundance of each product ion of interest from said second mass spectrum above said predetermined ion abundance; determining an ion ratio defined by the formula: ion ratio=(ion abundance of said product ion of interest in a mass spectrum having a higher collision energy)/(ion abundance of said product ion of interest in a mass spectrum having a lower collision energy); determining a mass-to-charge ratio of an ion of interest having a high ion ratio; and comparing the obtained mass-to-charge ratio to a predetermined mass-to-charge ratio value associated with a known 3′ terminal base sequence, wherein the mass-to-charge ratio of the ion of interest being substantially equal to the predetermined mass-to-charge ratio value associated with a known 3′ terminal base sequence provides an indication of the 3′ terminal base sequence.

In one embodiment, the method further includes: ranking said ion ratios for each product ion of interest from a lowest to a highest ion ratio; and comparing the mass-to-charge ratio of at least the product ion of interest having the highest ion ratio in said ranking.

In one embodiment, the method further includes at least one of the following: selecting said 3′ terminal base sequence from CC, (CT), (CA), TT, (TA), (GC), AA, (GT), (GA), or GG, and the predetermined value is selected from about 595, 610, 619, 625, 634, 635, 643, 650, 659, and 675, respectively; or selecting said 3′ terminal base sequence from TCC, T(CT), TTT, T(CA), T(TA), T(GC), TAA, T(GT), T(GA), or TGG, wherein the parenthetical indicates the composition but not the order of the 3′ terminal base sequence.

In one embodiment, the method further includes: determining a total product ion area from said first mass spectrum; determining a product ion area for each product ion of interest from said first mass spectrum; determining the ion abundance of said product ion of interest in said first mass spectrum according to the formula: ion abundance=(product ion area/total product ion area of first mass spectrum); determining a total product ion area from said second mass spectrum; determining a product ion area for each product ion of interest from said second mass spectrum; determining an ion abundance of said product ion of interest in said second mass spectrum according to the formula: ion abundance=(product ion area/total product ion area of second mass spectrum); and wherein said ion ratio is determined according to the formula: ion ratio=((Product ion area)/(Total product ion area of the mass spectrum having the higher collision energy)/((Product ion area)/(Total product ion area of the mass spectrum having the lower collision energy)). The product ion area/total product ion area can be calculated as described herein.

In one embodiment, the present invention provides a method of sequencing a parent nucleic acid having a known mass. Such a method includes: obtaining a first tandem mass spectrum of the parent nucleic acid using a first collision energy; determining a mass-to-charge ratio of a smallest [w] ion from said mass spectrum so as to provide a known partial 3′ terminal base sequence; adding a first hypothetical base to said smallest [w] ion to provide a hypothetical second smallest [w] ion; determining a hypothetical mass-to-charge ratio of said hypothetical second smallest ion; and comparing the hypothetical mass-to-charge ratio of said hypothetical second smallest ion to the actual mass-to-charge ratios obtained from said first tandem mass spectrum, wherein when said first hypothetical base causes the hypothetical mass-to-charge ratio of the hypothetical second smallest [w] ion to be substantially equal to an actual mass-to-charge ratio in said spectrum, said parent nucleic acid has a sequence that comprises the first hypothetical base plus the known partial 3′ terminal base sequence.

In one embodiment, the method further includes: adding a second hypothetical base to said second smallest [w] ion to provide a hypothetical third smallest [w] ion; determining a hypothetical mass-to-charge ratio of said hypothetical third smallest [w] ion; and comparing the hypothetical mass-to-charge of said hypothetical third smallest ion to the actual mass-to-charge ratios obtained from said first tandem mass spectrum, wherein when said second hypothetical base causes the hypothetical mass-to-charge ratio of said hypothetical third smallest [w] ion to be substantially equal to an actual mass-to charge ratio in said spectrum, said sequence of the parent nucleic acid comprises the second hypothetical base plus the first hypothetical base plus the known partial 3′ terminal base sequence.

In one embodiment, the method further includes: determining an ion abundance of each product ion of interest from said first mass spectrum above a predetermined ion abundance; obtaining a second tandem mass spectrum using a second collision energy that is different from the first collision energy; determining an ion abundance of each product ion of interest from said second mass spectrum above said predetermined ion abundance; determining an ion ratio defined by the formula: ion ratio=(ion abundance of said product ion of interest in a mass spectrum having a higher collision energy)/(ion abundance of said product ion of interest in the mass spectrum having the lower collision energy); and ranking said ion ratios for each product ion of interest from a lowest to a highest ion ratio so that the hypothetical smallest ion [w] has an ion ratio that is no less than the median of all ion ratios for each product ion of interest.

In one embodiment, the present invention includes a method for sequencing a nucleic acid, which includes: obtaining a mass spectrum of the nucleic acid using a first collision energy; determining a first partial 3′ terminal base sequence of said nucleic acid from a first [w] ion; combining a first internal ion from said mass spectrum to said first [w] ion to provide a first hypothetical [w] ion; determining a first hypothetical mass-to-charge ratio of said first hypothetical [w] ion, wherein the charge of the first hypothetical [w] ion is one; comparing the first hypothetical mass-to-charge ratio of said first hypothetical [w] ion to actual mass-to-charge ratios obtained from said mass spectrum, wherein when said internal ion causes said first hypothetical mass-to-charge ratio of said first hypothetical [w] ion to be substantially equal to an actual mass-to-charge ratio in said spectrum, said nucleic acid has a sequence that comprises the first hypothetical internal ion plus the first partial 3′ terminal base sequence.

In one embodiment, the method further includes: determining a second partial 3′ terminal base sequence of said nucleic acid from a second [w] ion, said second partial 3′ terminal base sequence being different from said first partial 3′ terminal base sequence; combining a second internal ion from said mass spectrum to said second [w] ion to provide a second hypothetical [w] ion; determining a second hypothetical mass-to-charge ratio of said second hypothetical [w] ion; comparing the second hypothetical mass-to-charge ratio of said second hypothetical [w] ion to actual mass-to-charge ratios obtained from said mass spectrum, wherein when said second internal ion causes the second hypothetical mass-to-charge ratio of said second hypothetical [w] ion ratio to be substantially equal to an actual mass-to-charge ratio in said spectrum, said nucleic acid has a sequence that comprises the second internal ion plus the second partial 3′ terminal base sequence.

In one embodiment, the method further includes: determining a base composition between said first hypothetical [w] ion and said second hypothetical [w] ion by determining a mass difference between said first hypothetical [w] ion and said second hypothetical [w] ion; and optionally, verifying the 3′ terminal base sequence when the said first hypothetical [w] ion and said second hypothetical [w] ion are the same.

In one embodiment, the method further includes: determining a third 3′ terminal base sequence of said nucleic acid from a third [w] ion, wherein said third [w] ion is selected the group consisting of an actual [w] ion in said mass spectrum, said first hypothetical [w] ion, or said second hypothetical [w] ion; and wherein said third partial 3′ terminal base sequence is different from said first partial 3′ terminal base sequence and said second partial 3′ terminal base sequence; combining a third internal ion from said mass spectrum to said third [w] ion to provide a third hypothetical [w] ion; determining a third hypothetical mass-to-charge ratio of said third hypothetical [w] ion; and comparing the third hypothetical mass-to-charge ratio of said third hypothetical [w] ion to actual mass-to-charge ratios obtained from said mass spectrum, wherein when said third internal ion causes the third hypothetical mass-to-charge ratio of said third hypothetical [w] ion to be substantially equal to an actual mass-to charge ratio in said spectrum, said nucleic acid has a sequence that comprises the third internal ion plus the third partial 3′ terminal base sequence.

In one embodiment, the method further includes: at least one of the following: verifying the partial 3′ terminal base sequence by comparing the second partial 3′ terminal sequence and the third partial 3′ terminal base sequence; or verifying the 3′ terminal base sequence when the said first hypothetical [w] ion, said second hypothetical [w] ion, and said third hypothetical [w] ion are the same.

In one embodiment, the present invention provides a method for sequencing a nucleic acid, which includes: obtaining a first tandem mass spectrum of the nucleic acid using a first collision energy; determining a first partial 5′ terminal base sequence of said nucleic acid; combining a first internal ion from said mass spectrum with said 5′ terminal base sequence to form a first hypothetical [a-Base] ion; determining a first hypothetical mass-to-charge ratio of said first hypothetical [a-Base] ion; and comparing the first hypothetical mass-to-charge ratio of said first hypothetical [a-Base] ion to actual mass-to-charge ratios obtained from said mass spectrum, wherein when said first internal ion causes said first hypothetical mass-to-charge ratio of said first hypothetical [a-Base] ion to be substantially equal to an actual mass-to-charge ratio in said spectrum, said nucleic acid includes a sequence that comprises the first hypothetical internal ion plus the 5′ terminal base sequence.

In one embodiment, the method further includes: determining a second partial 5′ terminal base sequence of said nucleic acid, said second partial 5′ terminal base sequence being different from said first partial 5′ terminal base sequence; adding a second internal ion from said mass spectrum to said second partial 5′ terminal base sequence to provide a second hypothetical [a-Base] ion; determining a second hypothetical mass-to-charge ratio of said second hypothetical [a-Base] ion; comparing the second hypothetical mass-to-charge ratio of said second hypothetical [a-Base] ion to actual mass-to-charge ratios obtained from said mass spectrum, wherein when said second internal ion causes the second hypothetical mass-to-charge ratio of said second hypothetical [a-Base] ion to be substantially equal to an actual mass-to-charge ratio in said spectrum, said nucleic acid has a sequence that comprises the second internal ion plus the second partial 5′ terminal base sequence.

In one embodiment, the method further includes: at least one of the following: determining a base composition between said first hypothetical [a-Base] ion and said second hypothetical [a-Base] ion by determining a mass difference between said first hypothetical [a-Base] ion and said second hypothetical [a-Base] ion; or verifying the 5′ terminal base sequence when the said first hypothetical [a-Base] ion and said second hypothetical [a-Base] ion are the same.

In one embodiment, the method further includes: determining a third 5′ terminal base sequence of said nucleic acid; wherein said third partial 5′ terminal base sequence is different from said first partial 5′ terminal base sequence and said second partial 5′ terminal base sequence; adding a third internal ion from said mass spectrum to said third 5′ terminal base sequence to provide a third hypothetical [a-Base] ion; determining a third hypothetical mass-to-charge ratio of said third hypothetical [a-Base] ion; comparing the third hypothetical mass-to-charge ratio of said third hypothetical [a-Base] ion to actual mass-to-charge ratios obtained from said mass spectrum, wherein when said third internal ion causes the third hypothetical mass-to-charge ratio of said third hypothetical [a-Base] ion to be substantially equal to an actual mass-to charge ratio in said spectrum, said nucleic acid sequence comprises the third internal ion plus the third partial 5′ terminal base sequence.

In one embodiment, the method further includes: verifying the 5′ terminal base sequence when the said first hypothetical [a-Base] ion, said second hypothetical [a-Base] ion, and said third hypothetical [a-Base] ion are the same.

In one embodiment, the methods of sequencing a nucleic acid further include: obtaining a second tandem mass spectrum of said parent nucleic acid using a second collision energy that is different from said first collision energy; determining an ion abundance of each product ion of interest from said second mass spectrum above said predetermined ion abundance; and determining an ion ratio, wherein a low ion ratio is indicative of said product ion of interest having a high charge state, a high ion ratio is indicative of said product ion of interest having a low charge state, and an ion ratio near unity is indicative of said product ion of interest having an intermediate charge state, and wherein at least one internal ion has a low charge state.

These and other embodiments and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope.

FIG. 1 shows the 20-mer single stranded DNA (SEQ ID NO: 1) analyzed in the study. Cleavage points are labeled according to McLuckey's notation. See McLuckey et al., Tandem Mass Spectrometry of Small, Multiply Charged Oligonucleotides, J. Am. Soc. Mass Spectrom. 3 (1992) 60-70; McLuckey et al., Decompositions of multiply charged oligonucleotide anions, J. Am. Chem. Soc. 115 (1993) 12085-12095.

FIG. 2 shows the MS/MS spectrum of the 7⁻ charge state for the 20-mer used in the present invention. Amplified regions of product ions are shown in an attempt to assign charge state from isotopic peaks. Because the sequence is known, charge states are easily assigned. If this were an unknown, the charge state of the [a₄-A]⁻ product ion is the only product ion that could be assigned a charge state. Other product ion charge states would be uncertain.

FIG. 3 shows the MS/MS spectra of the 4⁻ charge state of the 20-mer at different collision energies. CE 16% (B) CE 19% and (C) CE 22%. Product ions discussed in the text are indicated and the structures are shown. Higher charge states predominate at low CE while singly charged ions predominate at higher CE.

FIG. 4 is a genealogy diagram outlining the major dissociation pathways for the 4⁻ charge state. Complements are bracketed, and they are used to validate charge state. Product ions formed via (A) [M-HA]⁴⁻ and (B) [M-HG]⁴⁻.

FIG. 5 demonstrates breakdown curves graphing product ion area/total ion area vs. CE for major product ions in FIG. 5. (A) 4⁻ product ions, (B) 3⁻ product ions, (C) 2⁻ product ions, and (D) 1⁻ product ions.

FIG. 6 is a genealogy diagram outlining the major dissociation pathways for the 5⁻ charge state. Complements are bracketed, and they are used to validate charge state. Product ions formed via (A) [M-HA]⁵⁻ and (B) [M-HG]⁵⁻.

FIG. 7 shows the sequence information gained by identifying the smallest [w] ion (m/z 634) and extending the sequence with consecutively larger [w] ions. The singly charged internal ion (m/z 779) is shown below the sequence box and aids in verifying the proposed sequence.

FIG. 8 shows sequencing using internal ions. (A) Singly charged internal ion m/z 1700 combined with the existing sequence of 634 Da to extend the sequence to a mass of 2486 Da. The sequence extension is verified with other internal singly charged ions: m/z 1387, m/z 1098 and m/z 785 (shown below the sequence box). (B) m/z 1412 combined with previously outlined sequence of 1276 Da for sequence extension to 2800 Da. (C) (SEQ ID NO: 2) and (D) (SEQ ID NO: 3) Extension of sequence with m/z 810 and m/z 1123 to form extended sequence of 3432 Da (SEQ ID NO: 2) and 4075 Da (SEQ ID NO: 3), respectively.

FIG. 9 is the sequence information gained by working with singly charged ions originating from the 5′ terminus. (A) Placement of m/z 1019 and resulting base composition (SEQ ID NO: 4). (B) Placement of m/z 1636 and resulting sequence and base composition (SEQ ID NO: 5). (C) Placement of m/z 770 and resulting sequence and base composition (SEQ ID NO: 6).

FIG. 10 is a flow diagram illustrating the overall sequence strategy. The 5′ sequencing and the 3′ sequencing can be performed in any order or simultaneously.

### DETAILED DESCRIPTION OF THE PREFERRED EMBODIMENTS

Generally, the present invention includes the use of ion ratios in two tandem mass spectrometry experiments to work around the problem of poor resolution. Previously, ion ratios have previously been used by the present inventors to determine fragmentation genealogy for peptides, carbohydrates, and pharmaceuticals, but has yet heretofore been used to determine nucleic acid sequences. See Bandu et al., STEP (Statistical Test of Equivalent Pathways) Analysis: A Mass Spectrometric Method for Carbohydrates and Peptides, Anal. Chem. 77 (2005) 5886-5893; Bandu et al., The STEP Method (Statistical Test of Equivalent Pathways): Application to Pharmaceuticals, Analyst 131 (2006) 268-274. In general, the Statistical Test of Equivalent Pathways (“STEP”) analysis uses ion abundances in two tandem mass spectrometry experiments to obtain genealogy information about product ions present in mass spectra. To obtain genealogy information, STEP ratios are calculated by comparing the relative abundances of product ions in two MS/MS experiments. For singly charged ions, these ratios are directly related to the origin of the product ions. Product ions that result directly from the precursor ion have STEP ratios that are less than or equal to unity. Ions that result from secondary fragmentation pathways have STEP ratios that are significantly larger than the primary ions, based on a Q test of statistical significance. Consequently, the type (primary or secondary) of all the product ions in an MS/MS experiment is identified, when all the ions are the same charge state. Although the STEP method was applied to sequencing of peptides and carbohydrates, the use of STEP to improve DNA sequencing using mass spectrometry has not been disclosed or investigated.

The present invention is directed to a novel method for sequencing nucleic acids (e.g., oligonucleotides of DNA or RNA) using mass spectrometry. The present invention uses ion ratios calculated from MS/MS (or MSⁿ) data of a nucleic acid to be sequenced. The ratios are calculated from ion abundances obtained in one high dissociation MS/MS (or MSⁿ) experiment and one low dissociation MS/MS (or MSⁿ) experiment. Because highly charged DNA dissociates readily to form product ions with lower charge states, the ion ratio-based method described can be used to determine charge state information for the product ions of DNA, as demonstrated herein. Obtaining charge state information is important in DNA sequencing because this information is frequently not obtainable from inspection of the mass spectra, but identifying charge states, particularly identifying singly charged ions, greatly facilitates assigning base compositions to MS peaks.

In another aspect, singly charged ions, can be used to sequence moderately sized DNA oligomers. In one aspect of the present invention, a 20-mer is sequenced herein. The ion ratio method described herein is directly applicable to the verification of known nucleic acid sequences, and it can be utilized to sequence unknown nucleic acids.

The present invention can be used to identify the sequence of nucleic acids of any length; however, the nucleic acids are preferably oligonucleotides having between 5 and 200 bases, and even more preferably between 10 and 100 bases, and sill more preferably between about 20 and 50 bases.

The present invention provides a method of using ion ratios in two tandem mass spectrometry experiments in order to overcome any poor resolution of the data. Additionally, the present invention utilizes a Statistical Test of Equivalent Pathways (“STEP”) analysis with ion abundances in two tandem mass spectrometry experiments in order to obtain genealogy information about product ions present in mass spectra. Also, the present invention obtains genealogy information by calculating STEP ratios by comparing the relative abundances of product ions in two MS/MS experiments. Furthermore, the present invention can identify the type (e.g., primary or secondary) of all the product ions in an MS/MS experiment. Additionally, the present invention provides novel methods for sequencing oligonucleotides using a single MS/MS experiment. The methods of the present invention are described in more detail below.

In one embodiment, the present invention includes a method of determining a charge state of a mass spectrometry product ion of interest that is derived from a parent nucleic acid. Such a method includes the following: obtaining a first tandem mass spectrum of said parent nucleic acid using a high collision energy; determining an ion abundance of said product ion of interest from said first mass spectrum; obtaining a second tandem mass spectrum using a low collision energy; determining an ion abundance of said product ion of interest from said second mass spectrum; determining an ion ratio of the first and second mass spectra; and determining the charge state of the ion of interest by the ion ratio of said ion of interest. A low ion ratio is indicative of said product ion of interest having a high charge state, a high ion ratio is indicative of said product ion of interest having a low charge state, and an ion ratio near unity (i.e., 1) is indicative of said product ion of interest having an intermediate charge state

In one embodiment, determining the ion ratio is performed by calculating the following formula: ion ratio=(Ion abundance of said product ion of interest in said first mass spectrum, when the first mass spectrum has the higher collision energy)/(Ion abundance of said product ion of interest in said second mass spectrum, when the second mass spectrum has the lower collision energy). Alternatively, the ion ratio=(Product ion area)/(Total ion area at high dissociation)/(Product ion area)/(Total ion area at low dissociation). Additionally, ion abundance can be expressed as (Product ion area)/(Total ion area at low dissociation) or via equations (2)/(1).

$$\begin{matrix}Total Product Ion Area = {\sum }_{Lowest m / z}^{2000} m / z relative abundance & ( 1 ) \\ Product Ion Area = {\sum }_{- 1.0}^{+ 1.0} m / z relative abundance ( ion of interest ) & ( 2 )\end{matrix}$$

In one embodiment, the parent nucleic acid has a charge state of about 5⁻. A low ion ratio of about 0.7 or less is indicative of the product ion of interest having a high charge state of 3⁻ or greater. A high ion ratio is greater than about 2 and is indicative of the product ion of interest having a single charge. An ion ratio near unity (i.e., 1) or between about 0.8 and about 1.5 is indicative of the product ion of interest having a intermediate charge state of 2⁻ or 1⁻.

In one embodiment, the total product ion area from the first mass spectrum range includes areas having a mass-to-charge ratio below about m/z 2000.

In one embodiment, the first collision energy reduces a most abundant product ion from 100% relative abundance to between about 70 to 95% relative abundance. The second collision energy reduces the most abundant product ion from 100% relative abundance to below about 50% relative abundance.

In one embodiment, the first collision energy has a high dissociation activation amplitude between about 24% to about 30%, and the second collision energy has a low dissociation activation amplitude between about 15% to about 20% on a quadrupole ion trap mass analyzer.

In one embodiment, the parent nucleic acid is an oligonucleotide having between about 10 and 50 bases.

In one embodiment, the product ion of interest has a relative abundance of more than about 20% of a most abundant ion in either the first mass spectrum on the second mass spectrum.

In one embodiment, the ion abundance of said product ion of interest is measured by a relative abundance of said product ion of interest such that the ion ratio is defined by (relative ion abundance of said product ion of interest in said mass spectrum that has the higher energy)/(relative ion abundance of said product ion of interest in second mass spectrum that has the lower energy).

In one embodiment, the present invention includes a method of determining a 5′ terminal base sequence of a parent nucleic acid having a known mass. Such a method includes the following: obtaining a first tandem mass spectrum of said parent nucleic acid using a high collision energy; determining an ion abundance of each product ion of interest from said first mass spectrum above a predetermined ion abundance; obtaining a second tandem mass spectrum of said parent nucleic acid using a low collision energy; determining an ion abundance of each product ion of interest from said second mass spectrum above said predetermined ion abundance; determining an ion ratio; and determining the 5′ terminal base sequence by determining the difference between the known mass of the parent nucleic acid and the mass of each product ion of interest having a low ion ratio and comparing that difference to a predetermined value associated with a known 5′ terminal base sequence.

In one embodiment, determining the ion ratio is performed by calculating the following formula: ion ratio=(Ion abundance of said product ion of interest in said first mass spectrum with the high collision energy)/(Ion abundance of said product ion of interest in said second mass spectrum with the low collision energy).

In one embodiment, the predetermined ion abundance is a relative ion abundance of about 20% of a most abundant ion.

In one embodiment, the low ion ratio is less than about 0.5.

In one embodiment, the method of determining a 5′ terminal base sequence of a parent nucleic acid having a known mass further includes ranking the ion ratios for each product ion of interest from a lowest to a highest ion ratio, then determining the mass difference between the parent nucleic acid and the product ion of interest having the lowest ion ratio in the ranking, and then comparing the difference to the predetermined value associated with the known 5′ terminal base sequence.

In one embodiment, the 5′ terminal base sequence comprises a single nucleotide selected from C, T. A, or G, and wherein said predetermined value is about 220, 225, 234, or 250 Da respectively.

In one embodiment, the step of determining the ion abundance of each product ion of interest from the first mass spectrum above the predetermined ion abundance includes: determining a total product ion area from the first mass spectrum; determining a product ion area for each product ion of interest from the first mass spectrum; and determining an ion abundance of the product ion of interest in the first mass spectrum according to ((product ion area)/(total product ion area of first mass spectrum)).

In one embodiment, the step of determining the ion abundance of each product ion of interest from the second mass spectrum above the predetermined ion abundance includes: determining a total product ion area from the second mass spectrum; determining a product ion area for each product ion of interest from the second mass spectrum; determining an ion abundance of the product ion of interest in the second mass spectrum according to ((product ion area)/(total product ion area of second mass spectrum)); and the ion ratio is therefore determined according to: Ion Ratio=((Product ion area)/(Total product ion area of first mass spectrum with the higher collision energy)/(Product ion area)/(Total product ion area of second mass spectrum with the lower collision energy)).

In one embodiment, the present invention includes a method of determining a 3′ terminal base sequence of a parent nucleic acid. Such a method includes: obtaining a first tandem mass spectrum of the parent nucleic acid using a high collision energy; determining an ion abundance of each product ion of interest from said first mass spectrum above a predetermined ion abundance; obtaining a second tandem mass spectrum using a low collision energy; determining an ion abundance of each product ion of interest from the second mass spectrum above the predetermined ion abundance; determining an ion ratio; and determining the 3′ terminal base sequence by determining the mass-to-charge ratio of ions of interest having a high ion ratio and comparing that to a predetermined value associated with a known 3′ terminal base sequence.

In one embodiment, determining the ion ratio is performed by calculating the following formula: ion ratio=((Ion abundance of said product ion of interest in said first mass spectrum having the higher collision energy)/(Ion abundance of said product ion of interest in said second mass spectrum having the lower collision energy)).

In one embodiment, the predetermined ion abundance comprises a relative ion abundance of about 20% of a most abundant product ion.

In one embodiment, the method of determining a 3′ terminal base sequence of a parent nucleic acid further includes the step of ranking the ion ratios for each product ion of interest from a lowest to a highest ion ratio, and the comparing step is first performed with a product ion of interest having the highest ion ratio in the ranking.

In one embodiment, the product ion of interest has a mass-to-charge ratio less than or equal to m/z 675.

In one embodiment, the 3′ terminal base sequence is selected from CC, CT, CA, TT, TA, GC, AA, GT, GA, or GG, and the predetermined value is selected from about 595, 610, 619, 625, 634, 635, 643, 650, 659, and 675, respectively.

In one embodiment, the 3′ terminal base sequence is selected from TCC, T(CT), TTT, T(CA), T(TA), T(GC), TAA, T(GT), T(GA), or T(GG), wherein the parenthetical indicates the composition but not the order of the 3′ terminal base sequence.

In one embodiment, the parent said nucleic acid is an oligonucleotide having between about 10 and 100 bases.

In one embodiment, the high ion ratio ranges between 1.5 and 6.

In one embodiment, the parent nucleic acid has a charge of 3⁻, 4⁻, 5⁻, 6⁻, 7⁻, 8⁻, 9⁻, or 10⁻.

In one embodiment, the step of determining an ion abundance of each product ion of interest from said first mass spectrum above said predetermined ion abundance comprises the steps of: determining a total product ion area from the first mass spectrum; determining a product ion area for each product ion of interest from the first mass spectrum; and determining an ion abundance of the product ion of interest in the first mass spectrum according to ((product ion area)/(total product ion area of first mass spectrum)).

In one embodiment, the step of determining the ion abundance of each product ion of interest from the second mass spectrum above the predetermined ion abundance comprises the steps of: determining a total product ion area from the second mass spectrum; and determining a product ion area for each product ion of interest from the second mass spectrum; and determining an ion abundance of the product ion of interest in the second mass spectrum according to ((product ion area)/(total product ion area of second mass spectrum)). The ion ratio is determined according to: Ion Ratio=((Product ion area)/(Total product ion area of first mass spectrum having the higher collision energy))/((Product ion area)/(Total product ion area of second mass spectrum having the lower collision energy)).

In one embodiment, the present invention includes a method of sequencing a parent nucleic acid having a known mass. Such a method includes: obtaining a first tandem mass spectrum of the parent nucleic acid using a first collision energy; determining the mass-to-charge ratio of a smallest [w] ion from the mass spectrum to provide a known partial 3′ terminal base sequence; adding a first hypothetical base to the smallest [w] ion to provide a hypothetical second smallest [w] ion; determining a hypothetical mass-to-charge ratio of the hypothetical second smallest ion; and comparing the hypothetical mass-to-charge ratio of the hypothetical second smallest ion to the actual mass-to-charge ratios obtained from the mass spectrum. When the first hypothetical base causes the hypothetical mass-to-charge ratio of the hypothetical second smallest [w] ion to be substantially equal to an actual mass-to-charge ratio in the spectrum, the sequence comprises the first hypothetical base plus the known partial 3′ terminal base sequence.

In one embodiment, the method of sequencing a parent nucleic acid having a known mass further comprises the steps of: adding a second hypothetical base to the second smallest [w] ion to provide a hypothetical third smallest [w] ion; determining a hypothetical mass-to-charge ratio of the hypothetical third smallest [w] ion; and comparing the hypothetical mass-to-charge of the hypothetical third smallest ion to the actual mass-to-charge ratios obtained from the mass spectrum. When the second hypothetical base causes the hypothetical mass-to-charge ratio of the hypothetical third smallest [w] ion to be substantially equal to an actual mass-to charge ratio in the spectrum, the sequence comprises the second hypothetical base plus the first hypothetical base plus the known partial 3′ terminal base sequence.

In one embodiment, the first hypothetical base is selected from the group consisting of A, T, C, and G.

In one embodiment, the parent nucleic acid is an oligonucleotide having between about 10 and 50 bases.

In one embodiment, the method of sequencing a parent nucleic acid having a known mass further comprises the steps of: determining an ion abundance of each product ion of interest from the first mass spectrum above a predetermined ion abundance; obtaining a second tandem mass spectrum using a low collision energy; determining an ion abundance of each product ion of interest from the second mass spectrum above the predetermined ion abundance; determining an ion ratio; and spectrum ranking said ion ratios for each product ion of interest from a lowest to a highest ion ratio so that the hypothetical smallest ion [w] has an ion ratio that is no less than the median of all ion ratios for each product ion of interest.

In one embodiment, determining the ion ratio is performed by calculating the following formula: ion ratio=((Ion abundance of said product ion of interest in said first mass spectrum having the higher collision energy)/(Ion abundance of said product ion of interest in said second mass having the lower collision energy).

In one embodiment, the present invention includes a method for sequencing a nucleic acid. Such a method includes: obtaining a mass spectrum of the nucleic acid using a first collision energy; determining a first partial 3′ terminal base sequence of the nucleic acid from a first [w] ion; combining a first internal ion from the mass spectrum to the first [w] ion to provide a first hypothetical [w] ion; determining a first hypothetical mass-to-charge ratio of the first hypothetical [w] ion wherein the charge of the first hypothetical [w] ion is one; and comparing the first hypothetical mass-to-charge ratio of the first hypothetical [w] ion to actual mass-to-charge ratios obtained from the mass spectrum. When the internal ion causes said first hypothetical mass-to-charge ratio of said first hypothetical [w] ion to be substantially equal to an actual mass-to-charge ratio in said spectrum, said sequence comprises the first hypothetical internal ion plus the first partial 3′ terminal base sequence.

In one embodiment, the method for sequencing a nucleic acid further includes: determining a second partial 3′ terminal base sequence of the nucleic acid from a second [w] ion, the second partial 3′ terminal base sequence being different from the first partial 3′ terminal base sequence; combining a second internal ion from the mass spectrum to the second [w] ion to provide a second hypothetical [w] ion; determining a second hypothetical mass-to-charge ratio of the second hypothetical [w] ion; and comparing the second hypothetical mass-to-charge ratio of the second hypothetical [w] ion to actual mass-to-charge ratios obtained from the mass spectrum. When the second internal ion causes the second hypothetical mass-to-charge ratio of the second hypothetical [w] ion ratio to be substantially equal to an actual mass-to-charge ratio in the spectrum, the sequence comprises the second internal ion plus the second partial 3′ terminal base sequence.

In one embodiment, the mass difference between the first hypothetical [w] ion and the second hypothetical [w] ion is used to determine a base composition between the first hypothetical [w] ion and the second hypothetical [w] ion.

In one embodiment, the method for sequencing a nucleic acid further includes verifying the 3′ terminal base sequence when the first hypothetical [w] ion and the second hypothetical [w] ion are the same.

In one embodiment, the method for sequencing a nucleic acid further includes: determining a third 3′ terminal base sequence of the nucleic acid from a third [w] ion, wherein the third [w] ion is selected from the group consisting of an actual [w] ion in the mass spectrum, the first hypothetical [w] ion, or the second hypothetical [w] ion; identifying the third partial 3′ terminal base sequence to be different from the first partial 3′ terminal base sequence and the second partial 3′ terminal base sequence; combining a third internal ion from the mass spectrum to the third [w] ion to provide a third hypothetical [w] ion; determining a third hypothetical mass-to-charge ratio of the third hypothetical [w] ion; and comparing the third hypothetical mass-to-charge ratio of said third hypothetical [w] ion to actual mass-to-charge ratios obtained from the mass spectrum. When the third internal ion causes the third hypothetical mass-to-charge ratio of the third hypothetical [w] ion to be substantially equal to an actual mass-to charge ratio in the spectrum, the sequence comprises the third internal ion plus the third partial 3′ terminal base sequence.

In one embodiment, the method for sequencing a nucleic acid further includes verifying the partial 3′ terminal base sequence by comparing the second partial 3′ terminal sequence and the third partial 3′ terminal base sequence.

In one embodiment, the method for sequencing a nucleic acid further includes verifying the partial 3′ terminal with the hypothetical sequence by comparing the second partial 3′ terminal base sequence and the third partial 3′ terminal base sequence.

In one embodiment, the method for sequencing a nucleic acid further includes verifying the 3′ terminal base sequence when the first hypothetical [w] ion, second hypothetical [w] ion, and third hypothetical [w] ion are the same.

In one embodiment, the present invention includes a method for sequencing a nucleic acid. Such a method includes: obtaining a first tandem mass spectrum of the nucleic acid using a first collision energy; determining a first partial 5′ terminal base sequence of the nucleic acid; combining a first internal ion from the mass spectrum with the 5′ terminal base sequence to form a first hypothetical [a-Base] ion; determining a first hypothetical mass-to-charge ratio of the first hypothetical [a-Base] ion; and comparing the first hypothetical mass-to-charge ratio of the first hypothetical [a-Base] ion to actual mass-to-charge ratios obtained from the mass spectrum. When the first internal ion causes the first hypothetical mass-to-charge ratio of the first hypothetical [a-Base] ion to be substantially equal to an actual mass-to-charge ratio in the spectrum, the sequence comprises the first hypothetical internal ion plus the 5′ terminal base sequence.

In one embodiment, the method for sequencing a nucleic acid further includes: determining a second partial 5′ terminal base sequence of the nucleic acid, the second partial 5′ terminal base sequence being different from the first partial 5′ terminal base sequence; adding a second internal ion from the mass spectrum to the second partial 5′ terminal base sequence to provide a second hypothetical [a-Base] ion; determining a second hypothetical mass-to-charge ratio of the second hypothetical [a-Base] ion; and comparing the second hypothetical mass-to-charge ratio of the second hypothetical [a-Base] ion to actual mass-to-charge ratios obtained from the mass spectrum. When the second internal ion causes the second hypothetical mass-to-charge ratio of the second hypothetical [a-Base] ion to be substantially equal to an actual mass-to-charge ratio in the spectrum, the sequence comprises the second internal ion plus the second partial 5′ terminal base sequence.

In one embodiment, the mass difference between the first hypothetical [a-Base] ion and the second hypothetical [a-Base] ion is used to determine a base composition between the first hypothetical [a-Base] ion and the second hypothetical [a-Base] ion.

In one embodiment, the method for sequencing a nucleic acid further includes verifying the 5′ terminal base sequence when the first hypothetical [a-Base] ion and the second hypothetical [a-Base] ion are the same.

In one embodiment, the method for sequencing a nucleic acid further includes: determining a third 5′ terminal base sequence of the nucleic acid; wherein the third partial 5′ terminal base sequence is different from the first partial 5′ terminal base sequence and the second partial 5′ terminal base sequence; adding a third internal ion from the mass spectrum to the third 5′ terminal base sequence to provide a third hypothetical [a-Base] ion; determining a third hypothetical mass-to-charge ratio of the third hypothetical [a-Base] ion; and comparing the third hypothetical mass-to-charge ratio of the third hypothetical [a-Base] ion to actual mass-to-charge ratios obtained from the mass spectrum. When the third internal ion causes the third hypothetical mass-to-charge ratio of the third hypothetical [a-Base] ion to be substantially equal to an actual mass-to-charge ratio in said spectrum, said sequence comprises the third internal ion plus the third partial 5′ terminal base sequence.

In one embodiment, the method for sequencing a nucleic acid further includes verifying the 5′ terminal base sequence when the first hypothetical [a-Base] ion, second hypothetical [a-Base] ion, and third hypothetical [a-Base] ion are the same.

Additionally, all of the methods described herein can be performed with obtaining mass spectra with higher collision energies and lower collision energies being obtained in any order. Additionally, the ion ratio can be calculated by: ion ratio=(ion abundance of said product ion of interest in a mass spectrum having a lower collision energy)/(ion abundance of said product ion of interest in a mass spectrum having a higher collision energy); however, this ion ratio can be considered an inverted ion ratio. When the ion ration is calculated in this manner it is an inverted ion ratio, thereby the ion ratio is obtained by: ion ratio=1/(inverted ion ratio). Alternatively, when the ion ratio is calculated in this manner, the methods are changed in that the high ion ratios described herein become low ion ratios, and the low ion ratios described herein become high ion ratios. Thus, the methods of the present invention described herein can be modified by calculating an inverted ion ratio and then calculating the ion ratio as described herein, or the inverted ion ratio can be used and the methods can be modified by converting all ion ratios to inverted ion ratios by: inverted ion ratio=1/(ion ratio). Such changes in the method of calculating the ion ratio are well within the skill of one of ordinary skill in the art.

### I. DEFINITIONS

Unless defined otherwise, all technical and scientific terms used herein have the same meaning as is commonly understood by one of skill in the art to which this invention belongs.

As used herein, the term “nucleic acid” embraces oligonucleotides or polynucleotides such as deoxyribonucleic acid (DNA) and ribonucleic acid (RNA) as well as analogs of either RNA or DNA, for example, made from nucleotide analogs, any of which are in single or double-stranded form. Nucleic acid molecules can be synthetic or can be isolated from a particular biological sample using any number of procedures which are well-known in the art, the particular procedure chosen being appropriate for the particular biological sample. The bases of DNA and RNA include purines, pyrimidines and purine and pyrimidine derivatives and modifications, which are linearly linked to a chemical backbone. Common chemical backbone structures are deoxyribose phosphate, ribose phosphate, and polyamide. The purines of both DNA and RNA are adenine (A) and guanine (G). Others that are known to exist include xanthine, hypoxanthine, 2- and 1-diaminopurine, and other more modified bases. The pyrimidines are cytosine (C), which is common to both DNA and RNA, uracil (U) found predominantly in RNA, and thymine (T) which occurs almost exclusively in DNA. Some of the more atypical pyrimidines include methylcytosine, hydroxymethyl-cytosine, methyluracil, hydroxymethyluracil, dihydroxypentyluracil, and other base modifications.

The method described herein is applicable to sequencing both modified and unmodified DNA sequences, as the key difference between these two possibilities is that the masses which are observed in each case is different. Since mass spectrometry can detect bases of any mass, it can be used to sequence an oligonucleotide of any base (or modified base) composition, as long as the modified bases are “searched for” in the sequencing process. For simplicity, the examples discussed below contain only the unmodified, typical, bases C, T, A, and G.

As used herein, the term “sample” embraces a composition containing the nucleic acids to be sequenced. In a preferred embodiment, the sample is a “biological sample” (i.e., any material obtained from a living source (e.g. human, animal, plant, bacteria, fungi, protist, virus)). The biological sample can be in any form, including solid materials (e.g. tissue, cell pellets and biopsies), and biological fluids (e.g. urine, blood, saliva, amniotic fluid and mouth wash (containing buccal cells)).

As used herein, the term “low collision energy” means the collision energy (“CE”) needed to reduce the most abundant product ion from 100% relative abundance to about 98, 96, 94, 92, 90, 88, 86, or 85% relative abundance. Typically, the “low collision energy” reduces the most abundant product ion to between about 98 and 80% abundance, with a preferred range typically between 95 and 90% relative abundance.

As used herein, the term “high collision energy” means the CE needed to further reduce this product ion to below about 50, 45, 40, or 35% relative abundance, and preferably below about 50% relative abundance.

As used herein, the term “parent nucleic acid” refers to a nucleic acid that may dissociate to form fragments during mass spectrum analysis. A parent nucleic acid is typically charged, and thus will form fragments that are charged.

As used herein, the term “product ion” or “daughter ion” refers to the product of a reaction during mass spectrometry analysis derived from a parent nucleic acid. Examples of product ions are [w] ions, [a-Base] ions, and internal ions.

The term “ion abundance” of an ion of interest as used herein embraces a measurement obtained from a mass spectrometer which correlates to the number of ions of interest. “Ion abundance” may be expressed in absolute terms (e.g. number of counts), relative terms (e.g. relative ion abundance), product ion area, or “normalized” (e.g. by determining the (product ion area)/(total product ion area) as discussed more fully below).

The term “ion ratio” broadly refers to the ratio of the ion abundance of an ion of interest in a first mass spectrum at a first collision energy to the ion abundance of an ion of interest in a second mass spectrum at a second collision energy. Thus, in one aspect the ion ratio may be defined as: ion ratio=(number of counts of the ion of interest in the first mass spectrum)/(number of counts of the ion of interest in the second mass spectrum). In another aspect, the ion ratio may be defined as: ion ratio=(relative abundance of the ion of interest in the first mass spectrum)/(relative abundance of the ion of interest in the second mass spectrum). In another aspect, the ion ratio may be defined as: ion ratio=(product ion area of the ion of interest in the first mass spectrum)/(product ion area of the ion of interest in the second mass spectrum). In yet another aspect, the ion ratio may be defined as set forth in Example 2.

### II. MASS SPECTROMETERS

Various mass spectrometers are known to those skilled in the art or later developed. The mass spectrometer may comprise an Electrospray, Atmospheric Pressure Photo Ionisation (“APPI”), Matrix Assisted Laser Desorption Ionisation (“MALDI”), Laser Desorption Ionisation (“LDI”), Fast Atom Bombardment (“FAB”), desorption electrospray ionization (“DESI”), desorption ionization on silica (“DIOS”), Liquid Secondary Ions Mass Spectrometry (“LSIMS”) ion source. Such ion sources may be provided with an eluent over a period of time, the eluent having been separated from a mixture by means of liquid chromatography or capillary electrophoresis. While Tandem mass spectrometry is described in connection with the present invention, other mass spectrometry techniques can be utilized in accordance with the present invention.

The mass analyzer preferably comprises a triple quadrupole mass analyzer, a Time of Flight (“TOF”) mass analyzer (an orthogonal acceleration Time of Flight mass analyzer is particularly preferred), a 2D (linear) or 3D (doughnut shaped electrode with two endcap electrodes) ion trap, a magnetic sector analyzer or a Fourier Transform Ion Cyclotron Resonance (“FTICR”) mass analyzer.

The fragmentation device may comprise a quadrupole rod set, an hexapole rod set, an octopole or higher order rod set or an ion tunnel comprising a plurality of electrodes having apertures through which ions are transmitted. The apertures are preferably substantially the same size. The fragmentation device may, more generally, comprise a plurality of electrodes connected to an AC or RF voltage supply for radially confining ions within the fragmentation device. An axial DC voltage gradient may or may not be applied along at least a portion of the length of the ion tunnel fragmentation device. The fragmentation device may be housed in a housing or otherwise arranged so that a substantially gas-tight enclosure is formed around the fragmentation device apart from an aperture to admit ions and an aperture for ions to exit from. A collision gas such as helium, argon, nitrogen, air, or methane may be introduced into the collision cell.

### III. CONTROL SYSTEM

A control system (not shown) provides control signals for the various power supplies (not shown) which respectively provide the necessary operating potentials for the ion source, ion guide, quadrupole mass filter, collision cell, and the mass analyzer. These control signals determine the operating parameters of the instrument, for example the mass to charge ratios transmitted through the mass filter and the operation of the analyzer. The control system may be a computer (not shown), which may also be used to process the mass spectral data acquired. The computer can also display and store mass spectra produced by the analyzer and receive and process commands from an operator. The control system may be automatically set to perform various methods and make various determinations without operator intervention, or may optionally require operator input at various stages.

The control system is also preferably arranged to alter the CE in the collision cell or other fragmentation device at a low dissociation CE or high CE as desired. In one mode, a relatively high voltage is applied to the collision cell which in combination with the effect of various other ion optical devices upstream of the collision cell is sufficient to cause a fair degree of fragmentation of oligonucleotides passing therethrough. In a second mode, a relatively low voltage is applied which causes relatively little significant fragmentation of oligonucleotides passing therethrough. The voltage used is instrument dependent, and it will vary dramatically particularly between instruments with different mass analyzers, but can be readily determined by those skilled in the art.

At the end of each experimental run, the data which has been obtained is analyzed and parent oligonucleotides and fragment ions can be recognized on the basis of the relative intensity of a peak in a mass spectrum obtained when the collision cell was in one mode compared with the intensity of the same peak in a mass spectrum obtained when the collision cell was in a second mode. Automation of the sequencing steps can then be performed in accordance with the steps outlined herein using programming techniques known to those skilled in the art. As such, the method steps of the present invention can be implemented into computer program products that can be used for sequencing nucleic acids as described herein.

### IV. AMPLIFICATION OF SEQUENCES

The nucleic acids of the present invention may be amplified, if necessary or desired, to increase the number of copies of the nucleic acids to be sequenced using, for example, polymerase chain reactions (PCR) technology or any of the amplification procedures. Amplification involves denaturation of template DNA by heating in the presence of a large molar excess of each of two or more oligonucleotide primers and four dNTPs (dGTP, dCTP, dATP, dTTP). The reaction mixture is cooled to a temperature that allows the oligonucleotide primer to anneal to nucleic acids to be sequenced, after which the annealed primers are extended with DNA polymerase. The cycle of denaturation, annealing, and DNA synthesis, the principal of PCR amplification, is repeated many times to generate large quantities of product which can be easily identified.

Although PCR is a reliable method for amplification of target sequences, a number of other techniques can be used such as ligase chain reaction, self sustained sequence replication, Q-beta replicase amplification, polymerase chain reaction linked ligase chain reaction, gapped ligase chain reaction, ligase chain detection, and strand displacement amplification.

### EXAMPLES

### Example 1

### Sample Preparation and Mass Spectrometric Analysis Sample Preparation

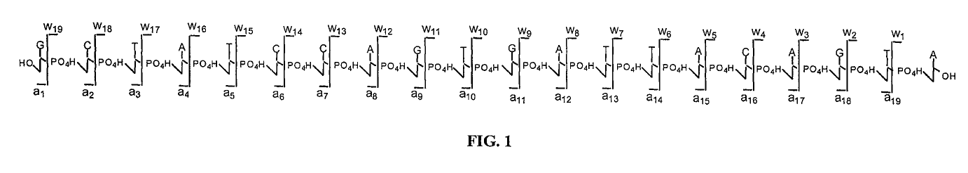

A 20-mer segment of single stranded DNA with a sequence of 5′-GCTATCCAGTGATTACAGTA-3′ (SEQ. ID NO. 1) was purchased from Midland Certified Reagent Company, Inc. (Midland, Tex.). The sequence is shown in FIG. 1. The sample was synthesized at Midland Certified Reagent Company, Inc. using cyanoethyl phosphoramidite chemistry and the product was purified using reversed phase HPLC. Approximately 221.7 nmol was initially dissolved in 1.0 mL HPLC grade water (Fisher Scientific, St. Louis, Mo.) and then diluted with HPLC grade methanol (Fisher Scientific, St. Louis, Mo.) to a concentration of approximately 20.0 μM.

### Mass Spectrometric Analysis

The DNA sample was introduced via syringe pump at a flow rate of 7 μL per minute. An LCQ Advantage quadrupole ion trap (Thermo Finnigan, San Jose, Calif.) was utilized for the analysis. This instrument has a mass range for detection between about 50-2000 m/z units and a mass accuracy of about 100 ppm (+/−0.1 amu). Tuning was performed to optimize the precursor ion signal in negative ion mode. The capillary temperature was set at 200° C., and the activation qz was set at 0.250. The capillary voltage was adjusted in the range of 2.8 to 3.3 kV to keep the spray current just under 1.0 μA. For MS/MS experiments, the parent ion was activated for 30 ms and an as isolation width of 4.0 Da was used. One high dissociation (MS/MS) spectrum and one low dissociation (MS/MS) spectrum were collected and 50 scans were averaged for data analysis. For this example, the low dissociation collision energy was defined as the collision energy (“CE”) needed to reduce the most abundant product ion from 100% relative abundance to about 90-95% relative abundance. For this example, the high dissociation CE was defined as the collision energy needed to further reduce this product ion to below 50% relative abundance. Determination of such dissociation collision energies is known to those skilled in the art.

### Comparative Example

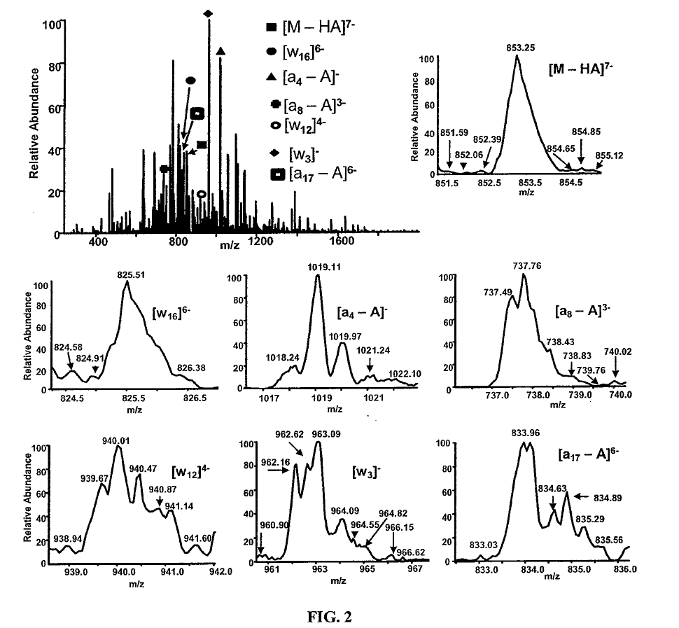

MS/MS data for DNA represent an especially complex data set, where even presumably simple tasks like charge state assignments for the product ions, are virtually impossible. The intrinsic nature of DNA, which makes it a fragile ion, leads to peak broadening, and a high spectral density for MS/MS data, where peaks with overlapping charge states are present. See Vachet et al., Ion-molecule reactions in a quadrupole ion trap as a probe of the gas-phase structure of metal complexes, J. Mass Spectrom. 33 (1998) 1209-1225; Murphy et al., Origin of mass shifts in the quadrupole ion trap: dissociation of fragile ions observed with a hybrid ion trap/mass filter instrument, Rapid Commun. Mass Spectrom. 14 (2000) 270-273; McClellan et al., Effects of fragile ions on mass resolution and on isolation for tandem mass spectrometry in the quadrupole ion trap mass spectrometer, Anal. Chem. 74 (2002) 402-412. FIG. 2 demonstrates examples of the difficulty in assigning charge states. It shows several product ions observed during MS/MS dissociation of the 20-mer described above at the 7⁻ charge state. Product ions formed through initial loss of the HA are labeled in the mass spectrum and their corresponding amplified regions of approximately 5 Da are included in the FIG. 2. Significant peaks within the 5 Da region are indicated to aid in attempts to assign charge state for these ions.

It will be appreciated to those skilled in the art that in this 7⁻ charge state example (and the 4⁻ and 5⁻ discussed in the following examples) of the 20-mer used dissociated via loss of neutral bases during MS/MS experiments, with adenine (A), guanine (G), and cytosine (C) losses observed. This is consistent with previous studies. See Pan et al., Investigation of the Initial Fragmentation of Oligodeoxynucleotides in a Quadrupole Ion Trap: Charge Level-Related Base Loss, J. Am. Soc. Mass Spectrom. 16 (2005) 1853-1865; Wan et al., Fragmentation mechanisms of oligodeoxynucleotides studied by H/D exchange and electrospray ionization tandem mass spectrometry, J. Am. Soc. Mass Spectrom. 12 (2001) 193-205. Product ions representing loss of thymine (T) were not observed to any significant extent, which was also previously observed. Further, loss of the neutral base is followed by backbone cleavage to form [w] and [a-Base] ions. See McLuckey et al., Tandem Mass Spectrometry of Small, Multiply Charged Oligonucleotides, J. Am. Soc. Mass Spectrom. 3 (1992) 60-70; McLuckey et al., Decompositions of multiply charged oligonucleotide anions, J. Am. Chem. Soc. 115 (1993) 12085-12095, which are incorporated by reference.

Since the sequence of this DNA is known, the peaks in the spectra shown in FIG. 2 can be assigned to specific compositions, and therefore, charge states can be inferred. However, if the sequence were unknown, many of these charge states would be ambiguous, both due to peak broadening and spectral complexity. Due to the low resolution of the QIT, product ions with higher charge states are not expected to be resolved ([M-HA]⁷⁻, [w₁₆]⁶⁻, [a₁₇-A]⁶⁻, [w₁₂]⁴⁻, and [a₈-A]³⁻), therefore, the charge state and effectively their masses, cannot be determined directly. Conversely, lower charge state assignments for product ions such as 1⁻ and 2⁻ should be resolved for charge state assignments, because ion traps have a resolution of 4000. See Siuzdak, The Expanding Role of Mass Spectrometry in Biotechnology, MCC Press, San Diego, 2003. As shown in FIG. 2, assigning a charge state to these product ions may also be difficult. The two singly charged ions in FIG. 2, [a₄-A]⁻ and [w₃]⁻, represent the difficulties associated with assigning even the 1⁻ charge state. The [a₄-A]⁻ ion does not have the optimal isotopic peak spacing of 1.0 Da, but the resolution provides enough information to assign a charge state with confidence. Conversely, the [w₃]⁻ ion, also singly charged, cannot definitively be assigned a charge state. There are two resolved peaks, 963.09 and 964.09 that are 1.0 Da apart, but overlapping signal is interfering and could cause ambiguity in the charge state assignment if this were an unknown sequence. The [w₃]⁻ (m/z 962) overlaps with another multiply charged ion with the same m/z value, [a₇-C]²⁻ demonstrating that, in addition to poor resolution, overlapping signal from multiple ions with the same m/z value is another contributor the inability to identify product ion charge state.

Overlapping signal is likely to be occurring with [a₁₇-A]⁶⁻ and [w₁₂]⁴⁻ as well. Both product ions have very large unresolved peaks of approximately 1 Da and additional significant peaks within a 2 Da range. The [a₁₇-A]⁶⁻ product ion is not characteristic of other highly charged product ions, such as [M-HA]⁷⁻ or [w₁₆]⁶⁻, but has a single, wide peak in the range from m/z 833-834 and then other significant peaks at m/z 834.63, m/z 834.29, and m/z 834.89. A similar observation can be noted for [w₁₂]⁴⁻ with a single, wide peak from m/z 939.67-940.47 and a secondary significant peak at m/z 941.14. Alternative peak assignments to account for the possible overlapping ions with the same m/z values could not be determined based on known fragmentation pathways, but this does not dismiss the possibility that overlapping product ion peaks are present. The peak shape for [a₁₇-A]⁶⁻ and [w₁₂]⁴⁻ is similar to the [w₃]⁻ product ion which is known to have overlapping signal, as discussed above. Overlapping signal of product ions with the same m/z values may possibly be identifiable due to the uncharacteristic peak shape as shown in FIG. 2, but even if this is possible, overlapping signal may still prevent identification of even the lower charge states. Overlapping signal has also been observed to cause interferences with sequencing by algorithms utilized in previous studies to determine the sequence of DNA, as discussed in latter sections. See Oberacher et al., Re-sequencing of multiple single nucleotide polymorphisms by liquid chromatography-electrospray ionization mass spectrometry, Nucleic Acids Res. 30 (2002) e67; Oberacher et al., Comparative Sequencing of nucleic acids by liquid chromatography-tandem mass spectrometry, Anal. Chem. 74 (2002) 211-218.

Of the seven ions shown in FIG. 2, only one product ion (e.g., [a₄-A]⁻) can be assigned a charge state directly and with confidence.

### Example 2

### Determination of Ion Ratio

This example demonstrates the determination of ion ratios used in the sequencing techniques of the present invention. For the analysis of the 4⁻ charge state, the low dissociation activation amplitude was 17%, as defined by the instrument software, and the high dissociation activation amplitude was 24%, as defined by the instrument software. For the 5⁻ charge state, the high dissociation and low dissociation activation parameters were 19% and 26%, respectively. This voltage is generally in the range of 0.8 to 1 V for the low activation conditions and 1.15 to 1.35 V for the high activation conditions on the quadrupole ion trap mass analyzer. Of course, it will be appreciated to those skilled in the art that there are many parameters that would affect the proper voltage to use including: activation time, collision gas, gas pressure, qz value, etc.

The spectrum lists for both the high and low dissociation experiments were imported into an Excel spreadsheet to perform calculations. However, a computer program product of the present invention could utilize the spectrum lists without import into another computer program. The ion ratios were calculated using a slightly modified version of the STEP method, as described in more detail earlier in conjunction with carbohydrates and peptides. See Bandu et al., STEP (Statistical Test of Equivalent Pathways) Analysis: A Mass Spectrometric Method for Carbohydrates and Peptides, Anal. Chem. 77 (2005) 5886-5893; Bandu et al., The STEP Method (Statistical Test of Equivalent Pathways): Application to Pharmaceuticals, Analyst 131 (2006) 268-274, which are incorporated by reference. The following equations were used to calculate the ion ratios:

$$\begin{matrix}Total Product Ion Area = {\sum }_{Lowest m / z}^{2000} m / z relative abundance & ( 1 ) \\ Product Ion Area = {\sum }_{- 1.0}^{+ 1.0} m / z relative abundance ( ion of interest ) & ( 2 ) \\ Ion Ratio = \frac{( Product ion area ) / ( Total ion area at high dissociation )}{( Product ion area ) / ( Total ion area at low dissociation )} & ( 3 )\end{matrix}$$

Equation (1) is a summation of all the m/z relative abundance values in the spectrum list. This value is defined as the Total Product Ion Area and is calculated for both the high dissociation and low dissociation MS/MS spectra.

Equation (2) is a summation of the relative abundance values in the spectrum list for individual product ions and is defined as the Product Ion Area. To calculate this value, the most abundant m/z in a product ion isotopic cluster was identified as the ion of interest in the MS/MS spectrum list. The relative values in the spectrum list are then summed for a 2 Da range that is centered on this ion of interest. The Product Ion Area is calculated for the ion of interest from both the high dissociation and the low dissociation MS/MS data.

Equation (3) is defined as the Ion Ratio. The Product Ion Area (equation (2)) calculated for the ion of interest from the high dissociation MS/MS data is divided by the calculated Total Ion Area (equation (1)) from the high dissociation MS/MS data. Similarly, the Product Ion Area (equation (2)) calculated for the ion of interest from low dissociation MS/MS data is divided by the calculated Total Ion Area (equation (1)) from the low dissociation MS/MS data. The Ion Ratio was calculated for all ions exceeding 20% relative abundance in either the high dissociation or the low dissociation spectra. It will be appreciated that the Ion Ratios may be determined for ions that are below or above the 20% relative abundance cut-off, which is used as an exemplary cut-off The threshold could be as low as 2% relative abundance, for example.

### Example 3

### Assignment of Charge States Using Ion Ratios

Before MS/MS data of DNA can be used for sequencing, a reliable method of assigning charge states, and effectively determining the mass of the product ions, must be available. McLuckey and co-workers have made significant contributions to this research area by identifying the major dissociation pathways for DNA and introducing the use of mass and charge conservation to identify complementary product ions in MS/MS and MSⁿ data. Complementary product ions are product ion pairs which, when their mass and charge are summed, equal the mass and charge of the precursor ion. See McLuckey et al., Tandem Mass Spectrometry of Small, Multiply Charged Oligonucleotides, J. Am. Soc. Mass Spectrom. 3 (1992) 60-70; McLuckey et al., Decompositions of multiply charged oligonlcleotide anions, J. Am. Chem. Soc. 115 (1993) 12085-12095, which are incorporated by reference.

For example, a precursor ion at the 4⁻ charge state could dissociate to form a complementary pair of product ions with 3⁻ and 1⁻ charge or a complementary pair of product ions with 2⁻ and 2⁻ charge. The total mass of these complementary pairs would be equivalent to the mass of the precursor ion. This method is an effective tool for indirectly identifying charge state assignments when poor resolution prevents direct assignments from isotopic peaks. The method is frequently utilized to verify known sequences, but was not designed to solve the problem of assigning spectra to large, unknown DNA sequences due to the complexity of the MS/MS data. In addition to spectral complexity, other factors also make the identification of complements, and therefore, determining charge state, difficult for larger unknown sequences. Product ions with high molecular weights and low charge states may not be trapped because they are outside the defined mass range (i.e. singly charged ions greater than 2000 Da). Product ions may also undergo subsequent dissociations at higher energies making their detection difficult.

In this example, McLuckey's complement method was used to validate a method of assigning charge states using an ion ratio approach. The ion ratio approach does not require any preconceived knowledge about a sequence, and it can accommodate larger oligomers, which produce MS/MS spectra that are too complex to fully assign using the complement method.

### Ion Ratios and Charge State

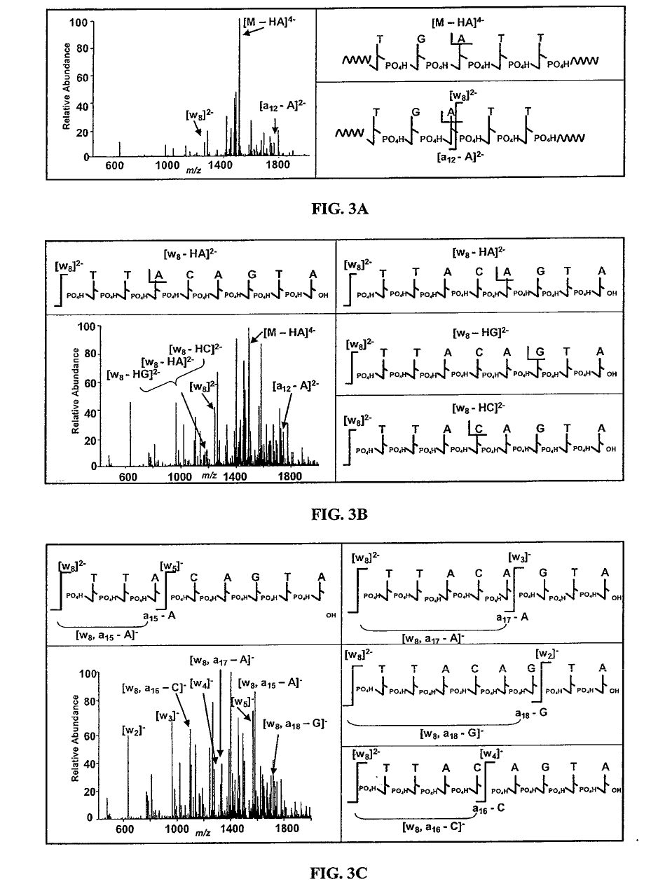

FIG. 3 shows the MS/MS spectra of the 4⁻ charge state of the 20-mer at three different collision energies: 16% (FIG. 3A); 19% (FIG. 3B), and 22% (FIG. 3C). FIGS. 3A to 3C demonstrate (1) how increasing activation voltage can affect the predominant ions present in a mass spectrum and (2) how this is related to the charge state of product ions. For simplicity, a single fragmentation pathway is outlined, the pathway involving the complementary product ion pair formed via loss of HA followed by backbone cleavage to form the [w₈]²⁻ and [a₁₂-A]²⁻ ions.

As shown in FIG. 3A, at low dissociation energy, the mass spectrum is dominated by ions with the 4⁻ charge state. The first product ion formed for the single dissociation pathway under discussion is the [M-HA]⁴⁻. Backbone cleavage ions, [w₈]²⁻ and [a₁₂-A]²⁻, are also observed, but to a much lesser extent (structures shown to the right). As shown in FIG. 3B, as the activation voltage is raised, ions with the 2⁻ charge state from this pathway are more pronounced ([w₈]²⁻ about 43% relative abundance and [a₁₂-A]²⁻ about 24% relative abundance, peaks are identified in the MS/MS spectrum). Additional product ions, formed through continued dissociation of the [w₈]²⁻ ion, are also present. These ions form by the secondary neutral loss of bases from the [w₈]²⁻ ion and are the [w₈-HG]²⁻, [w₈-HA]²⁻, and [w₈-HC]²⁻ ions (m/z 1167, m/z 1175, and m/z 1187, respectively, (structures are shown)). As shown in FIG. 3C, at even higher dissociation energy, the 4⁻ charge state ion ([M-HA]⁴⁻) has significantly decreased in intensity and the 2⁻ charge state ions ([w₈]²⁻, [a₁₂-A]²⁻, [w₈-HG]²⁻. [w₈-HA]²⁻, and [w₈-HC]²⁻) do not show significant increases or decreases in abundances. Singly charged [w] and internal ions formed through a secondary backbone cleavage predominate.

From these findings, it can be expected that ions with higher charge states will represent a smaller fraction of the total ion current, as the collision energy is increased. The present invention's ion ratio method measures this phenomenon by comparing an ion's contribution to the total ion current at low and high collision energy. When the ion's abundance decreases as collision energy increases, the ion ratio is less than one. Product ions with intermediate charge states have ion ratios of approximately one because their abundance does not change significantly with increasing activation. Lastly, singly charged ions undergo significant increases in abundance when the precursor ion is subject to higher activation conditions.

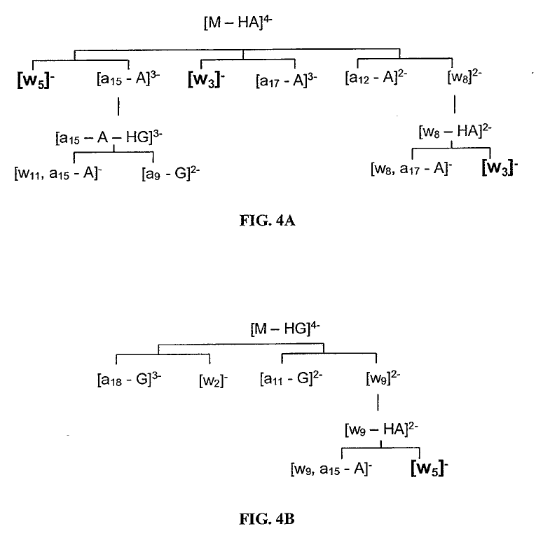

The [w₈]²⁻ and [a₁₂-A]²⁻ dissociation pathway discussed above and other major dissociation pathways are outlined in the genealogy diagram shown in FIG. 4. It will be appreciated that outlining all of the dissociation pathways at the high activation conditions used can be predicted and determined by one skilled in the art, and FIG. 4 represents an example only. The genealogy diagram was constructed using the complement method, where peaks were assigned using rules of “mass and charge conservation” outlined previously by McLuckey. The complements (indicated in brackets) are used to validate the charge state assignments herein.

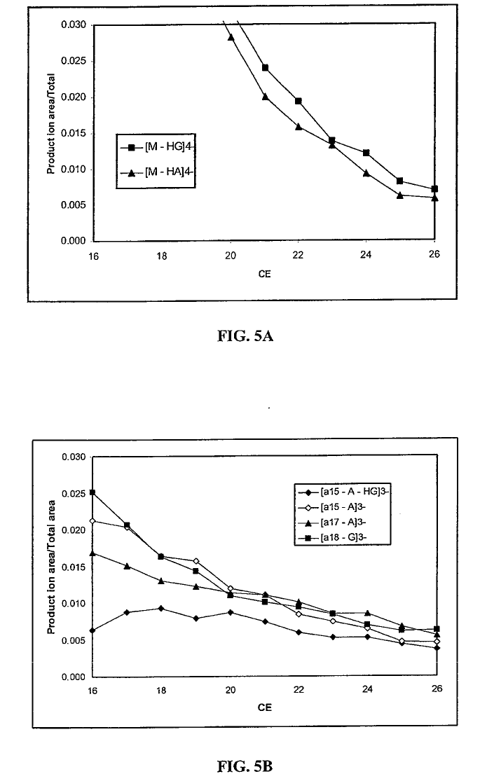

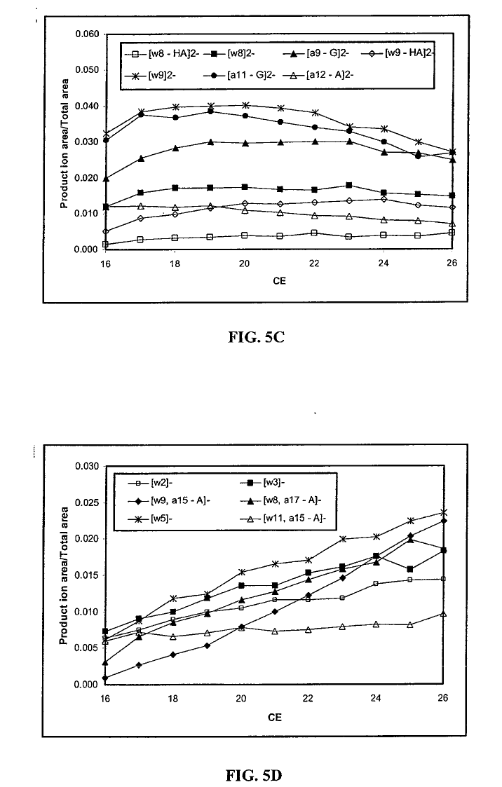

Breakdown curves graphing [product ion area/total ion area] vs. percent activation for the validated ions from FIG. 4, are shown in FIG. 5. FIG. 5 demonstrates that the charge state vs. activation trends observed for the [w₈]² and [a₁₂-A]²⁻ example (described above) are also observed for the other major product ions in the MS/MS spectrum. FIG. 5A demonstrates that other product ions formed through neutral loss of a base and retention of the 4⁻ charge, such as [M-HG]⁴⁻, have very steep breakdown curves. Product ions with a 3⁻ charge also decrease with increasing activation of the precursor, but to a much lesser extent since the slope in FIG. 5B is not as steep. FIG. 5C is a graph of 2⁻ product ions. Their ratios initially increase with increasing activation, but level off or slightly decrease at higher collision energies. Lastly, singly charged product ions, shown in FIG. 5D, increase substantially with increasing precursor ion activation. From these findings, one can conclude that ions having the same charge will have similar trends, when considering the ratio of percent product ion area with increasing precursor ion activation.

Table 1 summarizes data for the product ions shown in the genealogy diagram and the breakdown curves by listing the m/z value, ion ratio, and peak assignment. The product ions are listed by increasing ion ratios. From this table, it is apparent that ions with lower charge states located at the bottom right of Table 1, have higher ion ratios. Ions with higher charge states, located at the upper left of Table 1, have small ion ratios. Intermediate charge states have ion ratios closer to unity. This is also consistent with the findings demonstrated in FIG. 3. These results establish that ion ratios have a correlation with the charge state of product ions. Ordering the ions by ion ratio, in effect, orders product ions by charge state.

TABLE 1
4⁻ Charge State Product Ions

| m/z | ratio | Assignment | m/z | ratio | Assignment | m/z | ratio | Assignment |
|---|---|---|---|---|---|---|---|---|
| 1494.0 | 0.14 | [M − HA]⁴⁻ | 1745.2 | 0.82 | [a₁₂ − A]²⁻ | 634.1 | 1.70 | [w₂]⁻ |
| 1465.6 | 0.16 | [w₁₉]⁴⁻ | 1580.2 | 0.92 | [a₁₁ − G]²⁻ | 963.1 | 1.89 | [w₃]⁻ |
| 1489.9 | 0.16 | [M − HG]⁴⁻ | 1399.7 | 0.97 | [w₉]²⁻ | 1175.5 | 2.17 | [w₈ − HA]²⁻ |
| 1775.2 | 0.29 | [a₁₈ − G]³⁻ | 1242.7 | 1.05 | [w₈]²⁻ | 1565.1 | 2.37 | [w₅]⁻ |
| 1470.1 | 0.38 | [a₁₅ − A]³⁻ | 1263.2 | 1.05 | [a₉ − G]²⁻ | 1387.0 | 2.75 | [w₈, a₁₇ − A]⁻ |
| 1670.2 | 0.45 | [a₁₇ − A]³⁻ | 1731.1 | 1.23 | [w₁₁, a₁₅ − A]⁻ | 1098.1 | 7.56 | [w₉, a₁₅ − A]⁻ |
| 1419.8 | 0.71 | [a₁₅ − A − HG]³⁻ | 1332.1 | 1.59 | [w₉ − HA]²⁻ | | | |

From FIGS. 4 and 5, and the data in Table 1, the present invention illustrates how the ion ratio method can be used to organize nucleic acid (e.g. DNA) product ions by charge state. The general trend indicates that higher charge states have lower ion ratio values while singly charged ions have ion ratio values greater than one.

The ion ratios of Table 1, the genealogy diagram in FIG. 4, and the breakdown curves of FIG. 5 also show that the ion ratios organize species by charge state, and that this organization can be applied regardless of the ion's fragmentation pathway. As shown in FIG. 4, the [w₃]⁻ and the [w₅]⁻ product ions (in bold) can both be formed from more than one pathway. Although only a portion of the dissociation pathways are shown, formation of [w] and [a-Base] ions can potentially come from secondary backbone cleavage of all other higher [w] ions (w₁₉, w₁₆, w₁₂, etc.) and larger [a-Base] ions with higher charge states. The larger ratios for these singly charged ions (1.89 and 2.37) are probably the result of their formation through many pathways and minimal subsequent dissociation.

### Example 4

### Determination of 5′ Terminus Using Ion Ratios

This example demonstrates how the 5′ base may be easily identified by its small ion ratio. This information is very valuable in interpreting MS/MS data for sequencing unknowns which is demonstrated with the 5⁻ charge state

In general, after ranking the ion ratios from lowest to highest, the mass difference between the precursor ion and the first product ion in the list is determined. When the mass difference corresponds to a hypothetical base, the appropriate base is assigned.

As shown in Table 1, the [w₁₉]⁴⁻ ion listed in Table 1 has an ion ratio that is very low and similar in value to ions such as the [M-HA]⁴⁻ and [M-HG]⁴⁻ ions (0.16 vs. 0.14 and 0.16, respectively). The [w₁₉]⁴⁻ is important because it corresponds to loss of the 5′ base, so it can be used to identify the first nucleotide in the DNA sequence. The low ion ratio for [w₁₉]⁴⁻ makes this ion easy to identify, and identifying this ion is the first step of a protocol for sequencing an unknown oligomer, as described herein.

### Example 5

### Ion Ratios and the 5⁻ Charge State of the 20-Mer

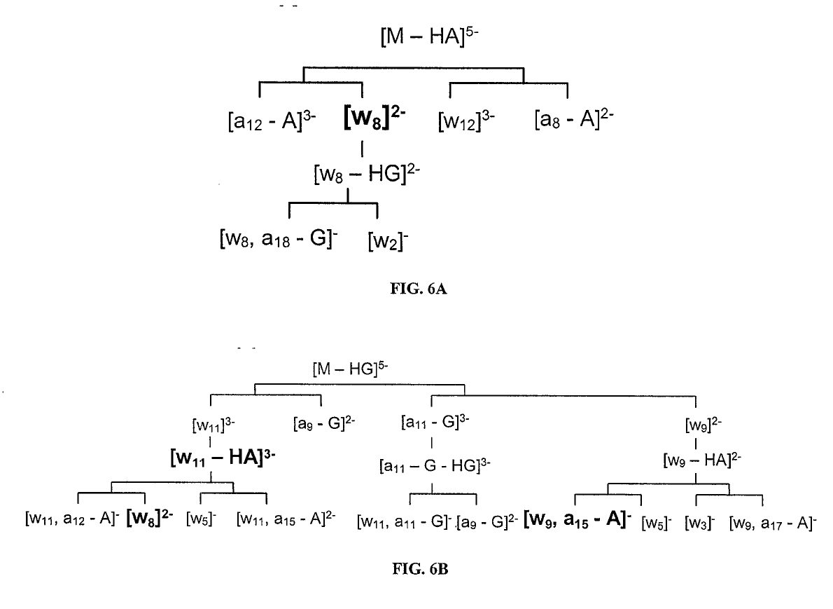

In this example, ion ratios for product ions apparent in the MS/MS data of the 5⁻ charge state of the 20-mer were analyzed to demonstrate the repeatability of the method to assign charge states, using a less abundant precursor ion. The data from this ion is also used to outline a sequencing strategy, which focuses on assigning the sequence from singly charged ions. FIG. 6 outlines the major fragmentation pathways for the 5⁻ charge state of the oligomer used in this research. The peaks were assigned using McLuckey's complement method. Table 2 lists all of the product ions exceeding 20% relative abundance in either the high dissociation or low dissociation MS/MS spectra of the 5⁻ charge state. Product ions are listed by increasing ion ratio. Peak assignments from the complement method and m/z values are also indicated. As discussed previously, outlining all the dissociation pathways may be determined by one skilled in the art, and thus, only the major pathways are outlined for exemplary purposes only.

Analysis of the 5⁻ charge state produces similar results to the 4⁻ charge state, where ion ratios increase with decreasing charge state as indicated in Table 2.

TABLE 2
5⁻ Charge State Product Ions

| m/z | ratio | Assignment | m/z | ratio | Assignment | m/z | ratio | Assignment |
|---|---|---|---|---|---|---|---|---|
| 1195.1 | 0.22 | [M − HA]⁵⁻ | 1263.6 | 1.12 | [a₉ − G]²⁻ | 1019.1 | 2.37 | |
| 1191.9 | 0.23 | [M − HG]⁵⁻ | 782.1 | 1.16 | | 1565.1 | 2.39 | [w₅]⁻ |
| 1172.2 | 0.25 | [w₁₉]⁵⁻ | 817.7 | 1.27 | | 1139.1 | 2.50 | |
| 1253.1 | 0.32 | [w₁₂]³⁻ | 887.5 | 1.27 | | 1021.6 | 2.59 | |
| 1164.9 | 0.34 | | 828.5 | 1.31 | | 1123.0 | 2.63 | |
| 1144.2 | 0.47 | [w₁₁]³⁻ | 1166.6 | 1.66 | [w₈ − HG]²⁻ | 982.7 | 2.86 | |
| 1162.7 | 0.47 | [a₁₂ − A]³⁻ | 1002.7 | 1.80 | [a₁₁ − G − HG]³⁻ | 785.1 | 2.99 | |
| 1053.2 | 0.54 | [a₁₁ − G]³⁻ | 849.6 | 1.81 | | 1636.1 | 3.01 | |
| 1142.1 | 0.58 | | 1387.1 | 1.82 | | 810.1 | 3.93 | [w₁₁, a₁₂ − A]⁻ |
| 933.1 | 0.62 | [w₉]³⁻ | 1098.1 | 1.93 | [w₉, a₁₅ − A]⁻, | 480.9 | 3.53 | [w₁₁, a₁₁ − G]⁻ |
| 1580.0 | 0.63 | [a₁₁ − G]²⁻ | | | [w₁₁ − HA]³⁻ | 1412.1 | 3.75 | |
| 1243.1 | 0.80 | [w₈]²⁻ | 634.1 | 1.94 | [w₂]⁻ | 865.1 | 3.77 | [w₁₁, a₁₅ − A]²⁻ |
| 1331.7 | 0.90 | [w₉ − HA]²⁻ | 1187.7 | 1.97 | | 770.0 | 3.86 | |
| 1399.7 | 0.93 | [w₉]²⁻ | 963.1 | 1.98 | [w₃]⁻ | 837.5 | 4.40 | |
| 1106.7 | 1.03 | [a₈ − A]²⁻ | 1323.0 | 2.02 | | 1700.1 | 4.57 | [w₈, a₁₈ − G]⁻, |
| | | | 1276.1 | 2.36 | | | | [w₉, a₁₇ − A]⁻ |
| | | | | | | 779.1 | 5.17 | |

A significant finding from the 5⁻ charge state data is the appearance of overlapping signals from two ions with the same m/z value but different charge states (indicated in bold, FIG. 6). In Table 2, this product ion (m/z 1098) is assigned as both the multiply charged [w₁₁-HA]³⁻ ion and the singly charged [w₉, a₁₅-A]⁻ ion. Although the charge state for these ions is different, the contribution of the singly charged ion to the product on area significantly increases the ion ratio to a value much greater than one (1.93). This result indicates that virtually no deviation from the expected value for singly charged ions is occurring with overlapping signal. Other singly charged ions with interferences from overlapping signal are expected to produce the same result, because singly charged ions increase the product ion area and do not dissociate to any significant extent. Lastly, as shown in bold on the genealogy diagram in FIG. 6, the origin of the product ion, again, has no effect on the ratios calculated. Similar to the 4⁻ charge state data, ions that can be formed from two different fragmentation pathways, do not have aberrant ion ratios. The product ion [w₈]²⁻ has a value similar to other ions at the 2-charge state (0.80) even though it could be formed by multiple pathways.

### Example 6

### Sequencing Unknown DNA with Singly Charged Ions

The ion ratio method is a robust approach to verifying known DNA sequences and sequencing unknowns, because it can assign MS/MS data, even when overlapping signal and secondary backbone cleavage is occurring. In fact, the ion ratio method for sequencing utilizes secondary backbone cleavage ions as part of the sequencing protocol, because these cleavages generate singly charged ions, which are the most straightforward for assigning base compositions.

In this example, the 5⁻ charge state of the 20-mer DNA sample was treated as an unknown to demonstrate how ion ratios can be utilized to sequence unknown DNA with singly charged ions. Product ion base composition assignment, and therefore, sequencing is more certain for singly charged ions. Also, singly charged ions are easy to identify in the ion ratio method because they have the largest ion ratios, as indicated in Table 2.

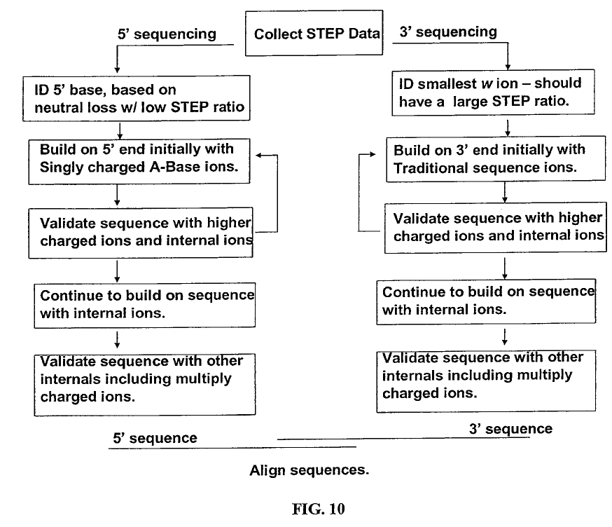

The sequencing strategies outlined below are similar to strategies developed by McCloskey and his colleagues for data obtained on the triple quadrupole mass spectrometer in the study described above. See Ni et al., Interpretation of oligonucleotide mass spectra for determination of sequence using electrospray ionization and tandem mass spectrometry, Anal. Chem. 68 (1996) 1989-1999. First, product ions containing the 3′ and/or 5′ base are identified. Then, the 3′ and/or 5′ ion is then extended to create longer oligomers. One major difference in McCloskey's sequencing strategy and the ion ratio sequencing strategy is that the ion ratio method utilizes singly charged internal ions to extend the sequence. In contrast, McCloskey's method extends the sequence with possible base compositions generated by the algorithm. Both methods verify the sequence with other multiply charged product ions in the MS/MS data, but the ion ratio method also verifies sequences with internal ions, shown in step 4 below. An overview of the sequencing steps in the present invention is shown in FIG. 10.

Step 1A: Identifying the 5′Base.

As discussed in the foregoing examples, the 5′ base can be identified as a G (m/z 1172.2=[w₁₉]⁵⁻) from the product ions with ratios much less than one (top of the ion ratio list in Table 2). As described above, the largest [w] ion has a low ion ratio, similar to product ions representing the neutral loss of a base from the precursor ion, such as [M-HA]⁵⁻ and [M-HG]⁵⁻. The base composition is identified by subtracting the mass of a phosphate, deoxyribose, hydroxy group, and one of the four possible bases from the precursor ion mass. Thus, starting at the top of Table 2 (lowest ion ratio), the mass difference between the precursor ion and the first product ion on the list is determined. If the value is about 220 (C), 225 (T), 234 (A), or 250 (G), the corresponding base is assigned.

Thus, in this example, the precursor mass was about 6116 Da. The mass of the ion was determined to be (1172.2 m/z×5 charge units)+5 protons=5865 Da. The difference is 6116 Da (precursor ion) minus 5865 Da (ion of interest)=251 Da. This corresponds to G as the appropriate 5′ base.

It will be appreciated that the m/z data from the mass spectra may be downloaded and incorporated in to a computer software program in order to automate the all or part of the data analysis discussed herein. Alternatively, the data analysis may be preformed manually.

Step 1B: Identifying the 3′ Base(s).

Singly charged ions (located at the bottom of ion ratio list) having m/z less than 675 (the mass of the largest dimer of bases) are utilized to identify the smallest [w] ion which corresponds to the to the 3′ base(s). If the m/z ratio corresponds to 595 (CC), 610 (CT), 619 (CA), 625 (TT), 634 (TA), 635 (GC), 643 (AA), 650 (GT), 659 (GA), 675 (GG), the appropriate base pair is assigned. It will be appreciated that the 3′ end consists of two bases (but the order is not known) because of resolution limits with mass spectrometer instruments. Improvements in mass spectrometers may make it possible to identify a single base.

In the event that none of the m/z ratios in the table correspond to an appropriate base using the foregoing method, this suggests that the 3′ base is a thymine (T), and the 3′ fragment consists of a thymine plus another base pair. Starting at the bottom of Table 2, the m/z ratios are then evaluated again. If the value is 899 (TCC), 914 (T(CT)), 929 (TTT), 923 (T(CA)), 938 (T(AT)), 939 (T(GC)), 947 (TAA), 954 (T(GT)), 963 (T(GA)), 979 (TGG), the corresponding triplet of bases is assigned.

In the event that none of the m/z ratios in the table correspond to an appropriate base using the foregoing method, this suggests that the third and fourth bases from the 3′ terminus are thymine (T), and the 3′ fragment consists of a two thymines plus another base pair. Starting at the bottom of Table 2, the m/z ratios are then evaluated again. If the value is 1203 (TTCC), 1218 (TT(CT)), 1233 (TTTT), 1227 (TT(CA)), 1242 (TT(AT)), 1243 (TT(GC)), 1251 (TTAA), 1258 (TT(GT)), 1267 (TT(GA)), 1283 (TTGG), the corresponding four bases are assigned.

In the event that none of the m/z ratios in the table correspond to an appropriate base using the foregoing method, this suggests that the third, fourth, and fifth bases from the 3′ terminus are thymine (T), and the 3′ fragment consists of three thymines plus another base pair. Starting at the bottom of Table 2, the m/z ratios are then evaluated again. If the value is 1507 (TTTCC), 1522 (TTT(CT)), 1537 (TTTTT), 1531 (TTT(CA)), 1546 (TTT(AT)), 1547 (TTT(GC)), 1555 (TTTAA), 1562 (TTT(GT)), 1571 (TTT(GA)), 1587 (TTTGG), the corresponding five bases are assigned.

In the event that m/z ratios correspond, the process can be repeated in a similar fashion with additional thymines added to the 3′ end. A predetermined cut-off may be optionally employed to terminate the sequence process.

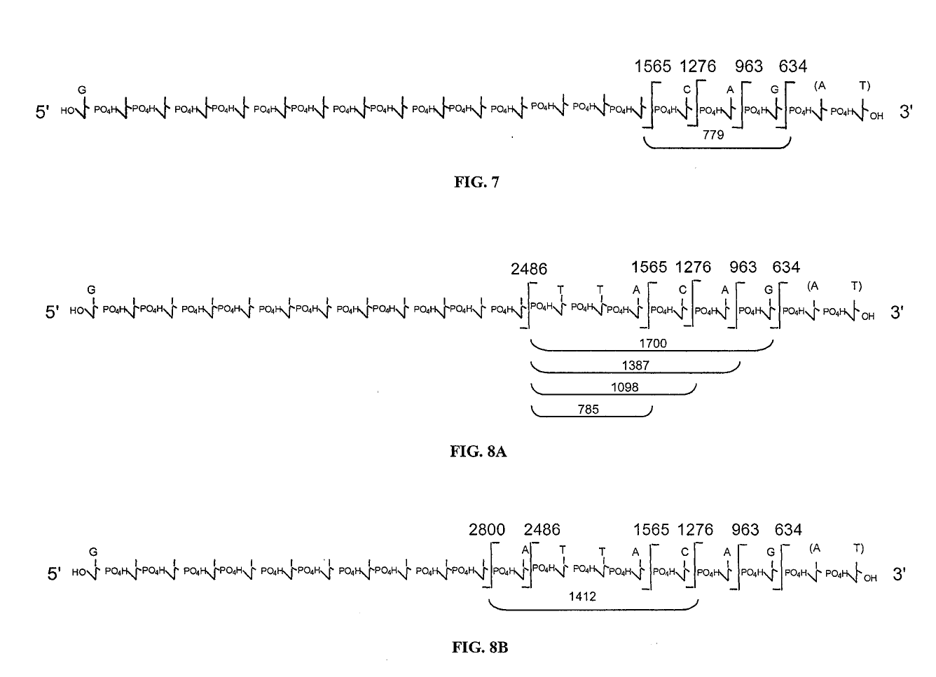

For example, starting from the bottom of the list of ions in Table 2, the product ion m/z 779.1 cannot be a [w] ion because no base combinations from the 3′ end can account for this singly charged ion. Other product ions at the bottom of Table 2, such as m/z 837.5 (ratio 4.40) and m/z 770.0 (ratio 3.86), are eliminated based on the same reasoning. The first possible [w] ion is m/z 634.1 (ratio 1.94). The ion has a base composition of A and T, but the order cannot be determined. FIG. 7 indicates the placement of the [w] ion at the 3′ terminus and the unknown order of the base composition AT is indicated in parenthesis.

Step 2: build on either ion identified in Step 1 by assigning consecutively higher [w] or [a-Base] ions.

To extend the sequence at the 5′ or 3′ ends, the [w] ions or the [a-Base] ions are built upon. For this example, the [w] ion outlined above ([w₂]⁻) is built upon. The next possible [w] ion must be greater than 923 Da. That is, the [w] ion comprises 634 Da from the smallest [w] ion plus at least 289 Da (from C, the smallest base) or a total of at least 923 Da. All ions below this threshold should preferably not be considered. Working up the column of ions present in Table 2, the mass difference between the [w] ion of interest and the smallest [w] ion is determined. If the value is about 289 (C), 304 (T), 313 (A), or 329 (G), the corresponding base is assigned.

In this particular example, the next possible [w] ion has an m/z 963.1 (ion ratio 1.98). The difference between m/z 963.1 (w ion of interest) and m/z 634.1 (smallest [w] ion) is 329 Da and is equivalent to G. Further, it is important note that no other base combinations have m/z values in Table 2:

m/z 634 ([w₂]⁻)+289 Da (C)=m/z 923 (not in Table 2)

m/z 634 ([w₂]⁻)+304 Da (T)=m/z 938 (not in Table 2)

m/z 634 ([w₂]⁻)+313 Da (A)=m/z 947 (not in Table 2)

m/z 634 ([w₂]⁻)+329 Da (G)=m/z 963 (in Table 2)

Thus, this extends the 3′ terminus of the sequence to 5′-G(AT)-3′.

Similarly, the next possible [w] ion is investigated. Starting at the bottom of Table 2 with the unidentified m/z values, the difference between m/z 1276 (w ion of interest) and m/z 963 ([w₃]⁻) is 313 Da, which is equivalent to A. Further, it is important note that no other base combinations have m/z values in Table 2:

m/z 963 ([w₃]⁻)+289 Da (C)=m/z 1252 (not in Table 2)

m/z 963 ([w₃]⁻)+304 Da (T)=m/z 1267 (not in Table 2)

m/z 963 ([w₃]⁻)+313 Da (A)=m/z 1276 (in Table 2)

m/z 963 ([w₃]⁻)+329 Da (G)=m/z 1292 (not in Table 2)

Thus, this extends the 3′ terminus of the sequence to 5′-AG(AT)-3′.

Similarly, the next possible [w] ion is investigated. Starting at the bottom of Table 2 with the unidentified m/z values, the difference between m/z 1565 (w ion of interest) and m/z 1276 (([w₃]⁻) is 289 Da which is equivalent to C. Further, it is important note that no other base combinations have m/z values in Table 2:

m/z 1276 ([w₄]⁻)+289 Da (C)=m/z 1565 (in Table 2)

m/z 1276 ([w₄]⁻)+304 Da (T)=m/z 1580 (not in Table 2)

m/z 1276 ([w₄]⁻)+313 Da (A)=m/z 1589 (not in Table 2)

m/z 1276 ([w₄]⁻)+329 Da (G)=m/z 1605 (not in Table 2)

Thus, this extends the 3′ terminus of the sequence to 5′-CAG(AT)-3′. Complete investigation of Table 2 for other [w] ions is shown in FIG. 7.

The next possible [w] ion must have a m/z of a least 1276 plus 289 Da (C) or m/z of 1854 Da. Thus, at this point, sequencing from the 3′ terminus stops using the foregoing techniques, and is continued using other sequencing strategies. However, singly charged internal ions, such as m/z 779.1, can also be identified in the ion ratio list and used to verify the proposed sequence (m/z 634 ([w₂]⁻)+2 protons+150 Da (G)+779 m/z (internal ion)=m/z 1565) as discussed more fully below. The internal ion is shown below the sequence box in FIG. 7.

Step 3: Combine [w] or [a-Base] ions previously outlined with singly charged internal ions to extend the sequence. Verify the new sequence assignments with multiply charged ions, and other singly charged internal ions.

Singly charged internal ions can be combined with the [w] ions outlined in FIG. 7 to extend the sequence. Importantly, extension of the sequence can optionally be expedited by considering only masses that have values greater than the largest [w] or [a-Base] ion. For example, extension of m/z 634.1 (the ([w₂]⁻) ion outlined in FIG. 7) with the singly charged ion m/z 837.5 would give a potential [w] ion 1471.6 Da. Because this combination of singly charged ions results in a mass less than 1565 Da, a [w] ion already identified in the sequence, this combination need not be considered.

From the bottom of the ion ratio list in Table 2, the sequence is extended using combinations of singly charged ions that combine to values greater than 1565 Da. Several attempts with various singly charged ions may be necessary to identify ions that correctly extend the sequence. Sequences are not verified as correct until other multiply charged ions confirm the assignment. Several examples of successful attempts at extending the sequence using this approach are described below.

Combining the singly charged ion at m/z 1700.1 to the existing [w] ion at m/z 634.1 presents a hypothetical [w] ion that is larger than the previously outlined [w] ions in FIG. 7. To identify the mass of the resulting hypothetical [w] ion from this extension, the dissociation events that form m/z 1700.1 must be considered. Internal ions, such as m/z 1700.1, are formed from two dissociation events, whereas only one cleavage produces a [w] ion. The second cleavage in the formation of internal ions results from an [a-Base] cleavage. To assign a mass to this hypothetical [w] ion and the extend sequence in the example described above (m/z 634.1+m/z 1700.1), the base lost in the [a-Base] cleavage (which produced the internal ion, m/z 1700.1) must be accounted for. The mass of the proposed sequence that is extended with m/z 1700.1 is 2486 Da. This is calculated by addition of the two singly charged ions, the base lost in the [a-Base] cleavage, and two protons (634 Da+1700 Da+150 Da (phosphate and sugar groups near cleaved G)+2 protons), as explained in the fragmentation mechanism for [a-Base] ions in McLuckey, Decompositions of multiply charged oligonucleotide anions, J. Am. Chem. Soc. 115 (1993) 12085-12095, which is incorporated by reference. Schematically, this is depicted as follows:

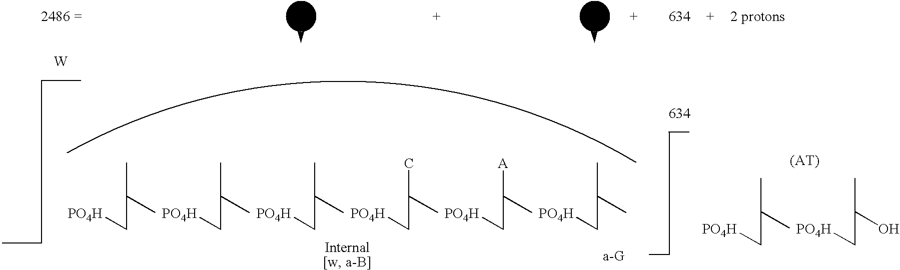

More generically, the m/z of the hypothetical [w] ion may be calculated as follows: m/z Hypothetical [w]=m/z internal ion [w, a-Base]+[Base]+2 protons+known [w] ion, wherein [Base] is equal to the neutral mass of the base lost, specifically, 150 Da for G; 110 Da for C; 134 Da for A and 125 Da for T.

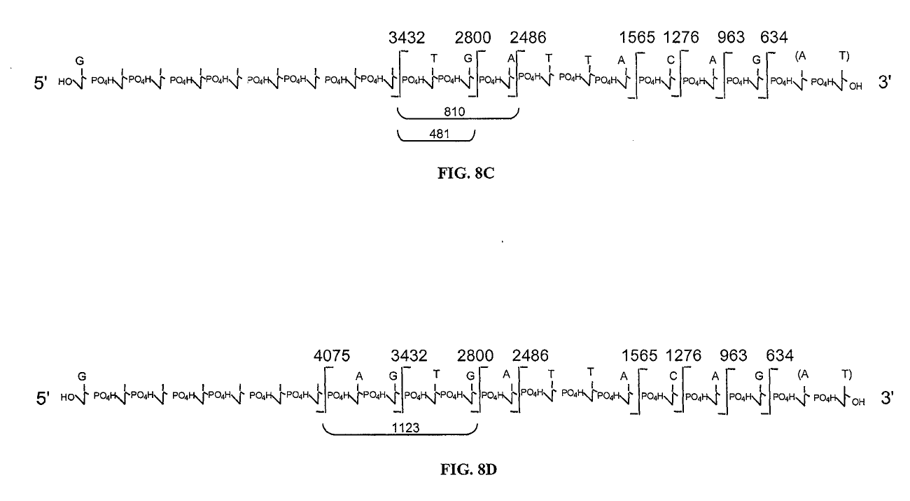

This hypothetical [w] ion at m/z 2486 contains three new bases (T, T, and A), which must be at the 5′ end of this ion. The assignment of TTA is made by subtracting the “known” bases from the internal ion, namely the C and A and the phosphate sugar backbone from the end residue, and assigning the rest of the mass to a base composition. For example: 1700−177 (the phosphate/sugar portion)−289 (C)−313 (A)=921. The mass of TTA is equal to (304*2)+313 or 921. It should be noted that TTA is the only set of three bases that adds up to the right mass of 921 Da so it is the only possible three base composition that could be assigned to this internal ion. Because DNA is more likely to dissociate by loss of A, the sequence for the three bases is 5′-TTA-3′. See Pan et al., Investigation of the Initial Fragmentation of Oligodeoxynucleotides in a Quadrupole Ion Trap: Charge Level-Related Base Loss, J. Am. Soc. Mass Spectrom. 16 (2005) 1853-1865; Wan et al., Fragmentation mechanisms of oligodeoxynucleotides studied by H/D exchange and electrospray ionization tandem mass spectrometry, J. Am. Soc. Mass Spectrom. 12 (2001) 193-205, which are incorporated by reference. The sequence can be verified by other internal ions as outlined in FIG. 8A below the sequence.

m/z 963 ([w₃]⁻)+2 protons+134 Da (A)+m/z 1387 (internal ion)=2486

m/z 1276 ([w₄]⁻)+2 protons+110 Da (C)+m/z 1098 (internal ion)=2486

m/z 1565 ([w₅]⁻)+2 protons+134 Da (A)+m/z 765 (internal ion)=2486

In addition, the doubly charged form of 2486 is m/z 1243.1. This ion is present in Table 2 and has a ratio (0.80), a value that is reasonable for a doubly charged ion. Thus, the ion ratios can be used to confirm DNA sequence.

In a similar manner, the singly charged internal ion, m/z 1412.1, can be combined with the [w] ion m/z 1276 ([w₄]⁻), resulting in a hypothetical sequence ion of 2800 Da (the doubly charged form is m/z 1399.7, ratio 0.93). The mass difference between consecutively greater [w] ions reveals the base composition between [w] ions as shown in FIG. 8B. FIG. 8B indicates that the base composition between ions having a mass of 2800 Da and 2486 Da can only be equal to an A (314 Da). While A is 313 Da, some small mass error is expected, due to the fact that larger oligomers will eventually be detected as their first isotopic peak (¹³C peak), instead of the monoisotopic mass. A sequencing program could account for this possibility, just as an investigator sequencing the oligonucleotide manually would also be aware of these mass differences.

The hypothetical [w] ions in FIGS. 8A and 8B that were verified by internal ions and doubly charged forms of ions can again be combined with other singly charged internal ions to make new hypothetical [w] ions that extend the sequence further. The extended sequence is verified with product ions having the 2⁻ or 3⁻ charged state. For example, m/z 810.1 combined with 2486 Da results in a hypothetical [w] ion with a mass of 3432 Da. That is, m/z 2486 ([w₅]⁻)+2 protons+134 (A)+m/z 810 (internal ion)=m/z 3432). The triply charged m/z value for this hypothetical [w] ion is m/z 1143.3 [(m/z 3432=2 protons)/3=1143.3)]. The ratio list has an ion m/z 1144.2 with a small ratio of 0.47, similar to other triply charged ions. The difference between the theoretical triply charge m/z value of 1143 and the observed product ion, m/z 1144.2, may be the result of overlapping signal from other product ions with the same m/z value that could potentially shift the m/z to a higher value. In addition, the monoisotopic mass will not be the largest isotope for this ion, so the fact that the “experimental” m/z value is larger than the theoretical value is not surprising. In this case, confidence in this potential [w] ion assignment can be improved by identifying m/z 480.9 which is an internal ion and is shown below the sequence in FIG. 8C. The mass difference between 3432 Da, the new hypothetical [w] ion, and the previously confirmed [w] ion of 2800 Da, corresponds to a base composition of TG. Because DNA is more likely to fragment by loss of G, the sequence between these two [w] ions is 5′-TG-3′. See Pan et al., Investigation of the Initial Fragmentation of Oligodeoxynucleotides in a Quadrupole Ion Trap Charge Level-Related Base Loss, J. Am. Soc. Mass Spectrom. 16 (2005) 1853-1865; Wan et al., Fragmentation mechanisms of oligodeoxynucleotides studied by H/D exchange and electrospray ionization tandem mass spectrometry, J. Am. Soc. Mass Spectrom. 12 (2001) 193-205, which are incorporated by reference. The extended sequence is shown in FIG. 8C.

FIG. 8D demonstrates the combination of an internal ion m/z 1123 to the [w] ion of mass 2800 Da to form another hypothetical [w] ion with a mass of 4075 Da. The difference between the hypothetical m/z 4075 [w] ion and the previously verified [w]ion of mass 3432 Da is equivalent to a base composition of AG. Although the triply charged form of this hypothetical [w] ion is not in Table 2 (it would appear at m/z 135.67, because the singly charged ion 4075 loses two protons to become 4073/3 or 1357.67). Verification of the base composition and sequence can be accomplished by proposing the sequence of 5′-AG-3′ or 5′-GA-3′ and identifying other ions that support one of these two assignments. To verify the sequence 5′-AG+3432 Da−3′, a triply charged [w] ion of 3761 Da should be in the ion ratio list. The theoretical triply charged form of this ion would be m/z 1252.7. The ion ratio list in Table 2 has a product ion m/z 1253.1 with an ion ratio of 0.32, a value acceptable for a triply charged ion. The difference between the theoretical m/z value and the measured m/z is likely to due to the fact that the monoisotopic mass would not be the largest peak in the isotope cluster for an ion this big, as previously noted. The extended sequence is shown in FIG. 8D.

Step 4: Extend the sequence with the remaining singly charged ions in the ion ratio list from the 5′ terminus.

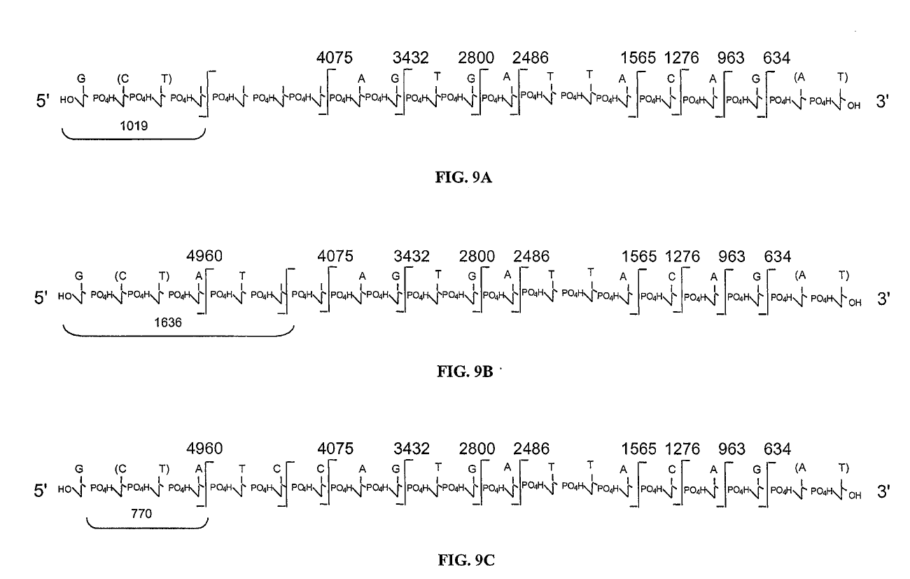

The remaining singly charged ions contain [a-Base] ions and internal ions of the form [w₁₉, a-B]⁻. Other ions, including internal ions that also lose a Base from their sequence, are also possibly present in the list. The smallest possible [a-Base] ion that includes the 5′ terminus is m/z 1019. The mass difference between the 5′ terminus and m/z 1019 corresponds to a base composition of (CT) but the order is unknown [G (250)+C (289)+T (304)+178 (which is the phosphate-sugar backbone that is also present in the cleavage)-2 protons=1019]. The two protons that are “lost” in this case are the same two protons that were added into the internal ion equation, discussed above. This extends the sequence to 5′-G(CT)−3′ as shown in FIG. 9A. In addition, the base lost prior to backbone cleavage is also unknown, which is also indicated in FIG. 9A. The next possible [a-Base] product ion is m/z 1636. The mass difference between m/z 1019 and m/z 1636 corresponds to the base composition AT, since A is (313 Da) and T is (304 Da). In this case, 1019+313+304=1636. No other combination of two bases adds to give a total of 617 (304+313) Da, so TA must be the two bases added to produce this ion. Because cleavage is more likely to occur at A, the sequence is 5′-ΔT-3′ and is shown in FIG. 9B. The mass of the [w] ion at the A cleavage point can now be determined and it is 4960 Da, also shown in FIG. 9B. The mass difference between this [w] ion and the largest [w] ion assigned from the 3′ end (4075 Da) corresponds to the mass of three bases with composition TCC. The final sequence is shown in FIG. 9C. The internal ion m/z 770 shown below the sequence verifies the proposed sequence. The method provides 80% sequence coverage. Put another way, of the 420 combinations possible, the sequence has been narrowed to four possibilities.

It will be appreciated that while the foregoing steps in the overall sequencing strategy are described in a particular order, modifications can be made to the order. For example, sequencing of the 3′ terminus can proceed before the 5′ terminus and vice versa.

### CONCLUSION

When analyzing DNA on the quadrupole ion trap, isotopic peaks, necessary for charge state assignments and effectively, product ion masses, are unresolved making sequencing of unknowns difficult. Multiple factors contribute to the inability to identify even singly charged ions. Major contributors include the poor resolution of the QIT, the fragility of DNA, which produces a high peak density, and overlapping signal from multiple ions with the same m/z value.

Ion ratios, previously used to characterize the structure of carbohydrates, peptides, and pharmaceuticals, are useful for ordering DNA product ions by charge state. DNA dissociation can be related to charge because product ions with higher charge states are more abundant at lower precursor activation energies and less abundant at higher activation energies than product ions at lower charge states. The ion ratio method takes advantage of this fact by using one high dissociation and one low dissociation MS/MS spectrum and ion abundances to identify the charge state of product ions.

Using ion ratios, singly charged ions can be easily identified. In addition, overlapping signal—from other multiply charged product ions with the same m/z value—is not a factor in identifying singly charged ions with the ion ratio method. Sequencing with singly charged ions provides the opportunity for greater confidence in mass assignments, which ultimately provides the ability to determine the base composition of product ions.

The sequencing strategy outlined here involves identifying 3′ and 5′ [w] and [a-Base] ions and extending the sequence with (1) other singly charged [w] and [a-Base] ions and (2) singly charged internal product ions. The proposed sequence can be verified by product ions with higher charge states and singly charged internal ions. The ion ratio method can be used to sequence unknowns as well as verify a known sequence.

Future research includes the analysis of additional unknowns, and will explore the limits of the ion ratio method for sequencing of larger DNA segments. In addition, expanding the method by incorporating computer algorithms for sequencing with singly charged ions, as well as multiply charged internal ions, may present additional opportunities for sequencing of larger segments. Attempts to utilize the method with MSⁿ experiments, which may improve the sequence coverage, are also under consideration.

### REFERENCES

The references cited here are incorporated by reference in their entirety as if each were individually incorporated by reference. The embodiments illustrated and discussed in this specification are intended only to teach those skilled in the art the best way known to the inventors to make and use the invention
B. Taback, D. S. B. Hoon, Circulating nucleic acids and proteomics of plasma/serum, Ann. N.Y. Acad. Sci. 1022 (2004) 1-8.
B. Taback, D. S. B. Hoon, Circulating nucleic acids in plasma and serum: past, present and future, Curr. Opin. Mol. Ther. 6 (2004) 273-278.W.
Y. Tong, Y. M. Dennis Lo, Diagnostic developments involving cell-free (circulating) nucleic acids, Clin. Chim. Acta 363 (2006) 187-196.
Y. M. Dennis Lo, Circulating nucleic acids in plasma and serum: an overview, Ann. N.Y. Acad. Sci. 945 (2001) 1-7.
S. Zeerleder, B. Zwart, W. A. Wuillemin, L. A. Aarden, A. B. J. Groeneveld, C. Caliezi, A. E. M. van Nieuwenhuijze, G. J. van Mierlo, A. J. M. Eerenberg, B. Lämmle, C. E. Hack, Elevated nucleosome levels in systemic inflammation and sepsis, Crit. Care Med. 31 (2003) 1947-1951.
S. Holdenrieder, P. Stieber, L. Y. S. Chan, S. Geiger, Cell-free DNA in serum and plasma: comparison of ELISA and quantitative PCR, Clin. Chem. 51 (2005) 1544-1546.
S. Holdenrieder, P. Stieber, H. Bodenmüller, M. Busch, G. Fertig, H. Futrst, A. Schalhorn, N. Schmeller, M. Untch, D. Seidel, Nucleosomes in serum of patients with benign and malignant diseases, Int. J. Cancer (Pred. Oncol.) 95 (2001) 114-120.
B. C. K. Wong, Y. M. Dennis Lo, Cell-free DNA and RNA in plasma as new tools for molecular diagnostics, Expert Rev. Mol. Diagn. 3 (2003) 785-797.
R. T. Cursons, E. Jeyerajah, J. W. Sleigh, The use of polymerase chain reaction to detect septicemia in critically ill patients, Crit. Care Med. 27 (1999) 937-940.
B. E. E. Cleven, M. Palka-Santini, J. Gielen, S. Meembor, M. Kronke, O. Krut, Identification and characterization of bacterial pathogens causing bloodstream infections by DNA microarray, J. Clin. Micro. 44 (2006) 2389-2397.
P. D. Wagner, M. Verma, S. Srivastava, Challenges for biomarkers in cancer detection, Ann. N.Y. Acad. Sci. 1022 (2004) 9-16.
E. Nordhoff, F. Kirpekar, P. Roepstorff, Mass spectrometry of nucleic acids, Mass Spectom. Rev. 15 (1996) 67-138.
K. K. Murray, DNA sequencing by mass spectrometry, J. Mass Spectrom. 31 (1996) 1203-1215.
J. H. Banoub, R. P. Newton, E. Esmans, D. F. Ewing, G. Mackenzie, Recent Developments in mass spectrometry for the characterization of nucleosides, nucleotides, oligonucleotides, and nucleic acids, Chem. Rev. 105 (2005) 1869-1915.
C. G. Huber, H. Oberacher, Analysis of nucleic acids by on-line liquid chromatography-mass spectrometry, Mass Spectrom. Rev. 20 (2001) 310-343.
C. C. Harris, p53: At the crossroads of molecular carcinogenesis and risk assessment, Science 262 (1993) 1980-1981.
E. Culotta, D. E. Koshland, Jr. p53 sweeps through cancer research, Science 262 (1993) 1958-1961.
A. J. Levine, p53, the cellular gatekeeper for growth and division, Cell 88 (1997) 323-331.
W. T. Muhammad, K. F. Fox, A. Fox, W. Cotham, M. Walla, Electrospray ionization quadrupole time-of-flight mass spectrometry and quadrupole mass spectrometry for genotyping single nucleotide substitutions in intact polymerase chain reaction products in K-ras and p53, Rapid Commun. Mass Spectrom. 16 (2002) 2278-2285.

J. J. Walters, W. Muhammad, K. F. Fox, A. Fox, D. Xie, K. E. Creek, L. Pirisi, Genotyping single nucleotide polymorphisms using intact polymerase chain reaction products by Electrospray quadrupole mass spectrometry, Rapid Commun. Mass Spectrom. 15 (2001) 1752-1759.
M. T. Krahmer, J. J. Walters, K. F. Fox, A. Fox, K. E. Creek, L. Pirisi, D. S. Wunschel, R. D. Smith, D. L. Tabb, J. R. Yates, III, MS for identification of single nucleotide polymorphisms and MS/MS for Discrimination of isomeric PCR products, Anal. Chem. 72 (2000) 4033-4040.

D. C. Muddiman, D. S. Wunschel, C. Liu, L. Pa\check{s}a-Tolić, K. F. Fox, A. Fox, G. A. Anderson, R. D. Smith, Characterization of PCR products from Bacilli using electrospray ionization FTICR mass spectrometry, Anal. Chem. 68 (1996) 3705-3712.
M. T. Krahmer, Y. A. Johnson, J. J. Walters, K. F. Fox, A. Fox, M. Nagpal, Electrospray quadrupole mass spectrometry analysis of model oligonucleotides and polymerase chain reaction products: determination of base substitutions, nucleotide additions/deletions, and chemical modifications, Anal. Chem. 71 (1999) 2893-2900.
D. S. Wunschel, D. C. Muddiman, K. F. Fox, A. Fox, R. D. Smith, Heterogeneity in Bacillus cereus PCR products detected by ESI-FTICR mass spectrometry, Anal. Chem. 70 (1998) 1203-1207.
B. M. Mayr, U. Kobold, M. Moczko, A. Nyeki, T. Koch, C. G. Huber, Identification of bacteria by polymerase chain reaction followed by liquid chromatography-mass spectrometry, Anal. Chem. 77 (2005) 4563-4570.
M. L. Metzker, Emerging technologies in DNA sequencing, Genome Res. 15 (2005) 1767-1776.
F. Sanger, S. Nicklen, A. R. Coulson, DNA sequencing with chain-terminating inhibitors, Proc. Natl. Acad. Sci. USA 74 (1977) 5463-5467.
D. P. Little, F. W. McLafferty, Sequencing 50-mer DNAs using Electrospray tandem mass spectrometry and complementary fragmentation methods, J. Am. Chem. Soc. 117 (1995) 6783-6784.
D. P. Little, D. J. Aaserud, G. A. Valaskovic, F. W. McLafferty, Sequence information from 42-108-mer DNAs (complete for a 50-mer) by tandem mass spectrometry, J. Am. Chem. Soc. 118 (1996) 9352-9359.
J. C. Schwartz, I. Jardine, Quadrupole ion trap mass spectrometry, Methods Enzymol. 270 (1996) 552-586.
A. Premstaller, K. Ongania, C. G. Huber, Factors determining the performance of triple quadrupole, quadrupole ion trap and sector field mass spectrometers in Electrospray ionization tandem mass spectrometry of oligonucleotides. 1. Comparison of performance characteristics, Rapid Commun. Mass Spectrom. 15 (2001) 1045-1052.
S. A. McLuckey, G. J. Van Berkel, G. L. Glish, Tandem Mass Spectrometry of Small, Multiply Charged Oligonucleotides, J. Am. Soc. Mass Spectrom. 3 (1992) 60-70.
M. L. Bandu, J. Wilson, R. W. Vachet, D. S. Dalpathado, H. Desaire, STEP (Statistical Test of Equivalent Pathways) Analysis: A Mass Spectrometric Method for Carbohydrates and Peptides, Anal. Chem. 77 (2005) 5886-5893.
M. L. Bandu, H. Desaire, The STEP Method (Statistical Test of Equivalent Pathways): Application to Pharmaceuticals, Analyst 131 (2006) 268-274.
S. Pan, K. Verhoeven, J. K. Lee, Investigation of the Initial Fragmentation of Oligodeoxynucleotides in a Quadrupole Ion Trap: Charge Level-Related Base Loss, J. Am. Soc. Mass Spectrom. 16 (2005) 1853-1865.
S. A. McLuckey, G. Vaidynathan, S. Habibi-Goudarzi, Charged vs. neutral nucleobase loss from multiply charged oligonucleotide anions, J. Mass Spectrom. 30 (1995) 1222-1229.
K. X. Wan, J. Gross, F. Hillenkamp, M. L. Gross, Fragmentation mechanisms of oligodeoxynucleotides studied by H/D exchange and electrospray ionization tandem mass spectrometry, J. Am. Soc. Mass Spectrom. 12 (2001) 193-205.
S. A. McLuckey, S. Habibi-Goudarzi, Decompositions of multiply charged oligonucleotide anions, J. Am. Chem. Soc. 115 (1993) 12085-12095.
R. W. Vachet, J. A. R. Hartman, J. H. Callahan, Ion-molecule reactions in a quadrupole ion trap as a probe of the gas-phase structure of metal complexes, J. Mass Spectrom. 33 (1998) 1209-1225.
J. P. Murphy, III, R. A. Yost, Origin of mass shifts in the quadrupole ion trap: dissociation of fragile ions observed with a hybrid ion trap/mass filter instrument, Rapid Commun. Mass Spectrom. 14 (2000) 270-273.
J. E. McClellan, J. P. Murphy, III, J. J. Mulholland, R. A. Yost, Effects of fragile ions on mass resolution and on isolation for tandem mass spectrometry in the quadrupole ion trap mass spectrometer, Anal. Chem. 74 (2002) 402-412.
G. Siuzdak, The Expanding Role of Mass Spectrometry in Biotechnology, MCC Press, San Diego, 2003.
H. Oberacher, P. J. Oefner, G. Hölzl, A. Premstaller, K. Davis, C. G. Huber, Re-sequencing of multiple single nucleotide polymorphisms by liquid chromatography-electrospray ionization mass spectrometry, Nucleic Acids Res. 30 (2002) e67.
H. Oberacher, B. Wellenzohn, C. G. Huber, Comparative Sequencing of nucleic acids by liquid chromatography-tandem mass spectrometry, Anal. Chem. 74 (2002) 211-218.
H. Oberacher, W. Parson, P. J. Oefner, B. M. Mayr, C. G. Huber, Applicability of tandem mass spectrometry to the automated comparative sequencing of long-chain oligonucleotides, J. Am. Soc. Mass Spectrom. 15 (2004) 510-522.
J. Ni, S. C. Pomerantz, J. Rozenski, Y. Zhang, J. A. McCloskey, Interpretation of oligonucleotide mass spectra for determination of sequence using electrospray ionization and tandem mass spectrometry, Anal. Chem. 68 (1996) 1989-1999.

From the foregoing it will be seen that this invention is one well adapted to attain all ends and objectives herein-above set forth, together with the other advantages which are obvious and which are inherent to the invention. Since many possible embodiments may be made of the invention without departing from the scope thereof, it is to be understood that all matters herein set forth or shown in the accompanying figures are to be interpreted as illustrative, and not in a limiting sense. While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope. All references (e.g., publications, journal articles, published patent applications, patents, and the like) recited herein are incorporated herein by specific reference in their entirety.

What is claimed is:

1. A method of determining a 5′ terminal base sequence of a parent nucleic acid having a known mass, the method comprising:
obtaining a first tandem mass spectrum of said parent nucleic acid using a first collision energy;
determining an ion abundance of each product ion of interest from said first mass spectrum above a predetermined ion abundance;
obtaining a second tandem mass spectrum of said parent nucleic acid using a second collision energy that is different from said first collision energy;
determining an ion abundance of each product ion of interest from said second mass spectrum above said predetermined ion abundance;
determining an ion ratio defined by the formula: ion ratio=(ion abundance of said product ion of interest in a mass spectrum having a higher collision energy)/(ion abundance of said product ion of interest in a mass spectrum having a lower collision energy);
determining a mass difference between the known mass of the parent nucleic acid and a mass of a product ion of interest having a low ion ratio; and
comparing the obtained mass difference to a predetermined mass value associated with a known 5′ terminal base sequence, wherein the mass difference provides an indication of the 5′ terminal base sequence.

2. The method of claim 1, wherein said 5′ terminal base sequence comprises a single nucleotide selected from C, T, A, or G, and wherein said predetermined mass value is about 220, 225, 234, or 250 Da, respectively, and wherein a mass difference substantially equal to the predetermined mass value of one of the known 5′ terminal base sequences indicates the 5′ terminal base sequence is the known 5′ base sequence.

3. The method of claim 1, further comprising:
ranking said ion ratios for each product ion of interest from a lowest to a highest ion ratio;
determining the mass difference between the parent nucleic acid and at least the product ion of interest having the lowest ion ratio in said ranking; and
comparing the mass difference to said predetermined mass value associated with the known 5′ terminal base sequence, wherein the mass difference being substantially the same as the predetermined mass value associated with the known 5′ terminal base sequence identifies the 5′ terminal base sequence of the parent nucleic acid.

4. The method of claim 1, further comprising:
identifying said ion ratios for each product ion of interest; and
determining the mass differences between the parent nucleic acid and selected product ions of interest from a lowest ion ratio to a highest ion ratio until obtaining a mass difference that is substantially the same as the predetermined mass value of one of the known 5′ terminal base sequences.

5. The method of claim 1, further comprising:
determining a total product ion area from said first mass spectrum;
determining a product ion area for each product ion of interest from said first mass spectrum;
determining the ion abundance of said product ion of interest in said first mass spectrum according to the formula: ion abundance=(product ion area/total product ion area of first mass spectrum);
determining a total product ion area from said second mass spectrum;
determining a product ion area for each product ion of interest from said second mass spectrum;
determining an ion abundance of said product ion of interest in said second mass spectrum according to the formula: ion abundance=(product ion area/total product ion area of second mass spectrum); and
wherein said ion ratio is determined according to the formula: ion ratio=((Product ion area)/(Total product ion area of the mass spectrum having the higher collision energy))/((Product ion area)/(Total product ion area of the mass spectrum having the lower collision energy)).

6. The method of claim 5, wherein the total product ion area and product ion area are calculated to:
$$\begin{matrix}Total Product Ion Area = {\sum }_{Lowest m / z}^{2000} m / z relative abundance & ( 1 ) \\ Product Ion Area = {\sum }_{- 1.0}^{+ 1.0} m / z relative abundance ( ion of interest ) & ( 2 )\end{matrix}$$

7. A method of determining a 3′ terminal base sequence of a parent nucleic acid, the method comprising:
obtaining a first tandem mass spectrum of said parent nucleic acid using a first collision energy;
determining an ion abundance of each product ion of interest from said first mass spectrum above a predetermined ion abundance;
obtaining a second tandem mass spectrum using a second collision energy that is different from said first collision energy;
determining an ion abundance of each product ion of interest from said second mass spectrum above said predetermined ion abundance;
determining an ion ratio defined by the formula: ion ratio=(ion abundance of said product ion of interest in a mass spectrum having a higher collision energy)/(ion abundance of said product ion of interest in a mass spectrum having a lower collision energy);
determining a mass-to-charge ratio of an ion of interest having a high ion ratio; and
comparing the obtained mass-to-charge ratio to a predetermined mass-to-charge ratio value associated with a known 3′ terminal base sequence, wherein the mass-to-charge ratio of the ion of interest being substantially equal to the predetermined mass-to-charge ratio value associated with a known 3′ terminal base sequence provides an indication of the 3′ terminal base sequence.

8. The method of claim 7, further comprising:
ranking said ion ratios for each product ion of interest from a lowest to a highest ion ratio; and
comparing the mass-to-charge ratio of at least the product ion of interest having the highest ion ratio in said ranking.

9. The method of claim 7, further comprising at least one of the following:
selecting said 3′ terminal base sequence from CC, (CT), (CA), TT, (TA), (GC), AA, (GT), (GA), or GG, and the predetermined value is selected from about 595, 610, 619, 625, 634, 635, 643, 650, 659, and 675, respectively; or
selecting said 3′ terminal base sequence from TCC, T(CT), TTT, T(CA), T(TA), T(GC), TAA, T(GT), T(GA), or TGG, wherein the parenthetical indicates the composition but not the order of the 3′ terminal base sequence.

10. The method of claim 7, further comprising:
determining a total product ion area from said first mass spectrum;
determining a product ion area for each product ion of interest from said first mass spectrum;
determining the ion abundance of said product ion of interest in said first mass spectrum according to the formula: ion abundance=(product ion area/total product ion area of first mass spectrum);
determining a total product ion area from said second mass spectrum;
determining a product ion area for each product ion of interest from said second mass spectrum;
determining an ion abundance of said product ion of interest in said second mass spectrum according to the formula: ion abundance=(product ion area/total product ion area of second mass spectrum); and
wherein said ion ratio is determined according to the formula: ion ratio=((Product ion area)/(Total product ion area of the mass spectrum having the higher collision energy)/((Product ion area)/(Total product ion area of the mass spectrum having the lower collision energy)).

11. The method of claim 10, wherein the total product ion area and product ion area are calculated to:
$$\begin{matrix}Total Product Ion Area = {\sum }_{Lowest m / z}^{2000} m / z relative abundance & ( 1 ) \\ Product Ion Area = {\sum }_{- 1.0}^{+ 1.0} m / z relative abundance ( ion of interest ) & ( 2 )\end{matrix}$$

12. A method of sequencing a parent nucleic acid having a known mass, the method comprising:
obtaining a first tandem mass spectrum of the parent nucleic acid using a first collision energy;
determining a mass-to-charge ratio of a smallest [w] ion from said mass spectrum so as to provide a known partial 3′ terminal base sequence;
adding a first hypothetical base to said smallest [w] ion to provide a hypothetical second smallest [w] ion;
determining a hypothetical mass-to-charge ratio of said hypothetical second smallest ion; and
comparing the hypothetical mass-to-charge ratio of said hypothetical second smallest ion to the actual mass-to-charge ratios obtained from said first tandem mass spectrum, wherein when said first hypothetical base causes the hypothetical mass-to-charge ratio of the hypothetical second smallest [w] ion to be substantially equal to an actual mass-to-charge ratio in said spectrum, said parent nucleic acid has a sequence that comprises the first hypothetical base plus the known partial 3′ terminal base sequence.

13. The method of claim 12, further comprising:
adding a second hypothetical base to said second smallest [w] ion to provide a hypothetical third smallest [w] ion;
determining a hypothetical mass-to-charge ratio of said hypothetical third smallest [w] ion; and
comparing the hypothetical mass-to-charge of said hypothetical third smallest ion to the actual mass-to-charge ratios obtained from said first tandem mass spectrum, wherein when said second hypothetical base causes the hypothetical mass-to-charge ratio of said hypothetical third smallest [w] ion to be substantially equal to an actual mass-to charge ratio in said spectrum, said sequence of the parent nucleic acid comprises the second hypothetical base plus the first hypothetical base plus the known partial 3′ terminal base sequence.

14. The method of claim 12, further comprising:
determining an ion abundance of each product ion of interest from said first mass spectrum above a predetermined ion abundance;
obtaining a second tandem mass spectrum using a second collision energy that is different from the first collision energy;
determining an ion abundance of each product ion of interest from said second mass spectrum above said predetermined ion abundance;
determining an ion ratio defined by the formula: ion ratio=(ion abundance of said product ion of interest in a mass spectrum having a higher collision energy)/(ion abundance of said product ion of interest in a mass spectrum having a lower collision energy); and
ranking said ion ratios for each product ion of interest from a lowest to a highest ion ratio so that the hypothetical smallest ion [w] has an ion ratio that is no less than the median of all ion ratios for each product ion of interest.

15. A method for sequencing a nucleic acid comprising:
obtaining a mass spectrum of the nucleic acid using a first collision energy;
determining a first partial 3′ terminal base sequence of said nucleic acid from a first [w] ion;
combining a first internal ion from said mass spectrum to said first [w] ion to provide a first hypothetical [w] ion;
determining a first hypothetical mass-to-charge ratio of said first hypothetical [w] ion, wherein the charge of the first hypothetical [w] ion is one;
comparing the first hypothetical mass-to-charge ratio of said first hypothetical [w] ion to actual mass-to-charge ratios obtained from said mass spectrum, wherein when said internal ion causes said first hypothetical mass-to-charge ratio of said first hypothetical [w] ion to be substantially equal to an actual mass-to-charge ratio in said spectrum, said nucleic acid has a sequence that comprises the first hypothetical internal ion plus the first partial 3′ terminal base sequence.

16. The method of claim 15, further comprising:
determining a second partial 3′ terminal base sequence of said nucleic acid from a second [w] ion, said second partial 3′ terminal base sequence being different from said first partial 3′ terminal base sequence;
combining a second internal ion from said mass spectrum to said second [w] ion to provide a second hypothetical [w] ion;
determining a second hypothetical mass-to-charge ratio of said second hypothetical [w] ion; and
comparing the second hypothetical mass-to-charge ratio of said second hypothetical [w] ion to actual mass-to-charge ratios obtained from said mass spectrum, wherein when said second internal ion causes the second hypothetical mass-to-charge ratio of said second hypothetical [w] ion ratio to be substantially equal to an actual mass-to-charge ratio in said spectrum, said nucleic acid has a sequence that comprises the second internal ion plus the second partial 3′ terminal base sequence.

17. The method of claim 16, further comprising:
determining a base composition between said first hypothetical [w] ion and said second hypothetical [w] ion by determining a mass difference between said first hypothetical [w] ion and said second hypothetical [w] ion; and
optionally, verifying the 3′ terminal base sequence when the said first hypothetical [w] ion and said second hypothetical [w] ion are the same.

18. The method of claim 16, further comprising:
determining a third 3′ terminal base sequence of said nucleic acid from a third [w] ion, wherein said third [w] ion is selected the group consisting of an actual [w] ion in said mass spectrum, said first hypothetical [w] ion, or said second hypothetical [w] ion; and wherein said third partial 3′ terminal base sequence is different from said first partial 3′ terminal base sequence and said second partial 3′ terminal base sequence;
combining a third internal ion from said mass spectrum to said third [w] ion to provide a third hypothetical [w] ion;
determining a third hypothetical mass-to-charge ratio of said third hypothetical [w] ion; and
comparing the third hypothetical mass-to-charge ratio of said third hypothetical [w] ion to actual mass-to-charge ratios obtained from said mass spectrum, wherein when said third internal ion causes the third hypothetical mass-to-charge ratio of said third hypothetical [w] ion to be substantially equal to an actual mass-to charge ratio in said spectrum, said nucleic acid has a sequence that comprises the third internal ion plus the third partial 3′ terminal base sequence.

19. The method of claim 18, further comprising at least one of the following:
verifying the partial 3′ terminal base sequence by comparing the second partial 3′ terminal sequence and the third partial 3′ terminal base sequence; or
verifying the 3′ terminal base sequence when the said first hypothetical [w] ion, said second hypothetical [w] ion, and said third hypothetical [w] ion are the same.

20. A method for sequencing a nucleic acid comprising:
obtaining a first tandem mass spectrum of the nucleic acid using a first collision energy;
determining a first partial 5′ terminal base sequence of said nucleic acid;
combining a first internal ion from said mass spectrum with said 5′ terminal base sequence to form a first hypothetical [a-Base] ion;
determining a first hypothetical mass-to-charge ratio of said first hypothetical [a-Base] ion; and
comparing the first hypothetical mass-to-charge ratio of said first hypothetical [a-Base] ion to actual mass-to-charge ratios obtained from said mass spectrum, wherein when said first internal ion causes said first hypothetical mass-to-charge ratio of said first hypothetical [a-Base] ion to be substantially equal to an actual mass-to-charge ratio in said spectrum, said nucleic acid includes a sequence that comprises the first hypothetical internal ion plus the 5′ terminal base sequence.

21. The method of claim 20, further comprising:
determining a second partial 5′ terminal base sequence of said nucleic acid, said second partial 5′ terminal base sequence being different from said first partial 5′ terminal base sequence;
adding a second internal ion from said mass spectrum to said second partial 5′ terminal base sequence to provide a second hypothetical [a-Base] ion;
determining a second hypothetical mass-to-charge ratio of said second hypothetical [a-Base] ion; and
comparing the second hypothetical mass-to-charge ratio of said second hypothetical [a-Base] ion to actual mass-to-charge ratios obtained from said mass spectrum, wherein when said second internal ion causes the second hypothetical mass-to-charge ratio of said second hypothetical [a-Base] ion to be substantially equal to an actual mass-to-charge ratio in said spectrum, said nucleic acid has a sequence that comprises the second internal ion plus the second partial 5′ terminal base sequence.

22. The method of claim 21, further comprising at least one of the following:
determining a base composition between said first hypothetical [a-Base] ion and said second hypothetical [a-Base] ion by determining a mass difference between said first hypothetical [a-Base] ion and said second hypothetical [a-Base] ion; or
verifying the 5′ terminal base sequence when the said first hypothetical [a-Base] ion and said second hypothetical [a-Base] ion are the same.

23. The method of claim 21, further comprising the step of:
determining a third 5′ terminal base sequence of said nucleic acid; wherein said third partial 5′ terminal base sequence is different from said first partial 5′ terminal base sequence and said second partial 5′ terminal base sequence;
adding a third internal ion from said mass spectrum to said third 5′ terminal base sequence to provide a third hypothetical [a-Base] ion;
determining a third hypothetical mass-to-charge ratio of said third hypothetical [a-Base] ion; and
comparing the third hypothetical mass-to-charge ratio of said third hypothetical [a-Base] ion to actual mass-to-charge ratios obtained from said mass spectrum, wherein when said third internal ion causes the third hypothetical mass-to-charge ratio of said third hypothetical [a-Base] ion to be substantially equal to an actual mass-to charge ratio in said spectrum, said nucleic acid sequence comprises the third internal ion plus the third partial 5′ terminal base sequence.

24. The method of claim 23, further comprising verifying the 5′ terminal base sequence when the said first hypothetical [a-Base] ion, said second hypothetical [a-Base] ion, and said third hypothetical [a-Base] ion are the same.

25. The method of claim 20, further comprising
obtaining a second tandem mass spectrum of said parent nucleic acid using a second collision energy that is different from said first collision energy;
determining an ion abundance of each product ion of interest from said second mass spectrum above said predetermined ion abundance; and
determining an ion ratio, wherein a low ion ratio is indicative of said product ion of interest having a high charge state, a high ion ratio is indicative of said product ion of interest having a low charge state, and an ion ratio near unity is indicative of said product ion of interest having an intermediate charge state, and wherein at least one internal ion has a low charge state.


SEQUENCE LISTING (6 sequences)

SEQ ID NO: 1 - DNA, 20 bp, Artificial
  Purchased from Midland Certified Reagent Company
gctatccagtgattacagta

SEQ ID NO: 2 - DNA, 11 bp, Artificial
  Portion of SEQ ID NO 1
tgattacagat

SEQ ID NO: 3 - DNA, 13 bp, Artificial
  Portion of SEQ ID NO 1
agtgattacagat

SEQ ID NO: 4 - DNA, 13 bp, Artificial
  Portion of SEQ ID NO 1
agtgattacagat

SEQ ID NO: 5 - DNA, 13 bp, Artificial
  Portion of SEQ ID NO 1
agtgattacagat

SEQ ID NO: 6 - DNA, 20 bp, Artificial
  Portion of SEQ ID NO 1
gctatccagtgattacagat